(19) United States
(12) Patent Application Publication
(10) Pub. No.: US 2013/0101632 A1
(43) Pub. Date: Apr. 25, 2013

(54) NANOPARTICULATE FORMULATIONS OF MITHRAMYCIN OR MITHRAMYCIN ANALOGUES FOR TREATING CANCER

(71) Applicant: University of Kentucky Research Foundation, Nashville, TN (US)

(72) Inventors: Daniel SCOTT, Lexington, KY (US); Jurgen ROHR, Georgetown, KY (US); Younsoo BAE, Lexington, KY (US)

(73) Assignee: UNIVERSITY OF KENTUCKY RESEARCH FOUNDATION, Nashville, TN (US)

(21) Appl. No.: 13/623,638

(22) Filed: Sep. 20, 2012

Related U.S. Application Data
(60) Provisional application No. 61/550,227, filed on Oct. 21, 2011.

(51) Int. Cl. A61K 31/704; A61P 35/00; C08G 73/08; A61K 9/14; A61K 31/785; B82Y 5/00

(52) U.S. Cl. 424/400; 514/33; 424/78.3; 525/420; 977/755; 977/788; 977/906; 977/773

(57) ABSTRACT

Methods and formulations for improving therapeutic potential of mithramycin (MTM) or MTM analogues are disclosed. For example, in certain aspects, methods for preparing a composition containing MTM or an MTM analogue nanoparticulate formulation and uses thereof are described. Furthermore, methods for delivering MTM or MTM analogues are disclosed.

### RELATED APPLICATIONS

This application claims priority to U.S. Provisional Patent Application No. 61/550,227, filed Oct. 21, 2011, the entire content of which is hereby incorporated herein by reference as though fully set forth here.

### GOVERNMENT LICENSE RIGHTS

The subject technology was made possible, in part, with Government support, and the U.S. Government has certain rights in the subject technology by the terms of grant Nos. R25CA153954 and CA091901 awarded by the National Institute of Health.

### FIELD

The subject technology relates in general to the field of drug delivery and medicinal chemistry; and more particularly, it concerns improvement of therapeutic potential of mithramycin and mithramycin analogues for treating cancer.

### BACKGROUND

Cancer causes about 13% of all deaths. According to the American Cancer Society, 7.6 million people died from cancer in the world during 2008. Although advances in treatment have been made, there continues to be a need for intervention strategies, including compounds that act as primary protective agents by preventing, delaying, or reversing preneoplastic lesions, as well as those that act on secondary or recurrent cancers as therapeutic agents. Mithramycin (MTM), a natural product of soil bacteria from the Streptomyces genus, displays potent anti-cancer activity but its use has been limited clinically by the severe side effects and toxicities it causes. Engineering of the MTM biosynthetic pathway has produced the 3-side chain modified analogues such as MTM SK (SK), MTM SDK (SDK) and MTM SA (SA), which have exhibited increased anticancer activity and improved therapeutic index. However, these analogues still suffer from low bioavailability, short plasma retention time and low intra-tumor accumulation.

Therefore, there remains a need in the medical arts for more efficient techniques for improving the solubility, stability, bioavailability and/or the pharmacokinetics of mithramycin or mithramycin analogues so that a better in vivo therapeutic activity can be achieved.

### SUMMARY

The inventors have previously discovered that SK and SDK analogues of MTM exhibit increased anticancer activity and improved therapeutic index over MTM itself. However, the therapeutic potential of SK or SDK is limited by the poor solubility, stability in solution, bioavailability and/or quick elimination in vivo.

One aspect of the present disclosure concerns a novel MTM or MTM analogue nanoparticulate formulation for cancer treatment to overcome these problems, including methods and procedures to prepare the nanoparticles of proper size distribution with defined amount of MTM or MTM analogue suitable for administration in vivo. This MTM or MTM analogue nanoparticulate formulation has beneficial pharmacological properties including improved solubility, stability, bioavailability, effective anticancer activity, and favorable tolerability in animals.

In one aspect, the subject technology relates to a nanoparticulate formulation including: (i) mithramycin (MTM) or one or more MTM analogues selected from the group consisting of SK (MTM with short side-chain and a ketone), SDK (MTM with short side-chain and diketone), SA (MTM with short side-chain and carboxylic acid) and a derivative thereof, as load; and (ii) block copolymer units of a polyamide and a biocompatible polymer as carrier. Chemical structures of MTM and exemplary MTM analogues are shown in FIGS. 8 and 9. In a related embodiment, the polyamide includes amino acid units derived from amino acids with side chains that can have positive, neutral, or negative charges. In another related embodiment, the biocompatible polymer is selected from the group consisting of poly(ethylene glycol) (PEG), chitosan (CS), polyethylenimine (PEI), poly(L-lactic acid) (PLLA), poly(D-lactic acid) (PDLA), poly(2-hydroxy ethyl methacrylate) (PHEMA), poly(ε-caprolactone) (PCL), poly(vinyl acetate) (PVAc), poly(ethylene oxide) (PEO), poly[(R)-3-hydroxybutyric acid)] (PHB), cellulose acetate (CA), poly(lactide-co-glycolide) (PLGA), poly(N-isopropylacrylamide) (PNIPAM), polyalkyleneglycol, polyalkyleneoxide, polyvinylpyrrolidone, polysaccharide, polyacrylamide, polymethacrylamide, polyvinylalcohol and derivatives thereof. In another related embodiment, the copolymer units form nanoparticles in form of supermolecular structures selected from the group consisting of micelles, vesicles, layers, membrane, sphere, aggregate, tube, fiber, ribbon and sheet. In a related embodiment, the block copolymer units self-assemble or crosslink to form a micelle with the polyamide forming the core and the biocompatible polymer forming the corona of the micelle. In another related embodiment, one or more of the amino acid units includes a pH-sensitive linkage in its side chain, wherein said pH-sensitive linkage is selected from the group consisting of acetal, orthoester, cis-aconityl group, hydrazone, imine, ester, Schiff base, dithioacetal, tert butyl ester, carbamate, thioester, or phosphoramidate. In another related embodiment, the amino acid units includes aspartic acid or glutamic acid or both. In another related embodiment, the MTM or one or more of the MTM analogues is conjugated to one or more of the amino acid units via the pH-sensitive linkage. In another related embodiment, the pH-sensitive linkage is stable at a pH between about 7 and 8 and is hydrolyzed at a pH less than about 7 to release the MTM or one or more of the MTM analogues. In another related embodiment, the block copolymer is poly(ethylene glycol)-poly(aspartate hydrazide) copolymer. In another related embodiment, the formulation is freeze dried to form a freeze-dried formulation.

In another aspect, the subject technology relates to a method of treating a hyperproliferative disease or a quiescent malignant disease including administering to a subject in need thereof a therapeutically effective amount of a nanoparticulate formulation of the subject technology, such as the one described above.

In another aspect, the subject technology relates to a method of decreasing the toxicity of MTM or one or more MTM analogues by incorporating the MTM or one or more of the MTM analogues in a formulation of the subject technology, such as the one described above.

The advantages and novel features are set forth in part in the description which follows, and in part will become apparent to those skilled in the art upon examination of the following and the accompanying drawings or may be learned by production or operation of the examples. The advantages of the present teachings may be realized and attained by practice or use of the methodologies, instrumentalities and combinations described herein.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 is a conceptual diagram illustrating self-assembled and cross-linked polymer micelles as nanoparticulate formulations for mithramycin (MTM) analogues.

FIG. 2 shows synthesis of block copolymers, cross-linked block copolymers, and drug conjugates.

FIG. 3 shows particle sizes of the drug containing self-assembled or cross-linked micelles in panel (A) and the drug entrapment yields in panel (B).

FIG. 4 shows drug release patterns from self-assembled and cross-linked micelles at pH 7.4 and 5.0 for 72 h. All data points are the average of three measurements±standard deviation. Error bars not visible are obstructed by the point.

FIG. 5 shows the cytotoxicity of free drug and nanoparticulate formulations for SK (panel A) and SDK (panel B) against A549 cell lines. All points are the average of 4 measurements±1 standard deviation. Error bars not visible are obstructed by the point.

FIG. 6 shows the effects of SK (panel A) and SDK (panel B) nanoparticulate formulations on cell viability in HT29 cells relative to free SK and SDK, respectively.

FIG. 7 shows the in vivo antitumor effects of SK (panel A) and SDK (panel B) nanoparticulate formulations in suppressing tumor volume in nude mice relative to free SK and SDK, respectively.

FIG. 8 shows the chemical structure of the MTM SA which can be further derivatized or substituted by various functional groups to form new MTM analogues with anti-cancer activity. The representative side chains depicted for substitution onto an MTM SA core are exemplary and are not to be construed as limiting the number of different MTM analogues that can possibly be produced for use in the methods and formulations of the present disclosure.

FIG. 9 shows the chemical structures of exemplary MTM analogues derivatives of MTM SA synthesized.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details.

Mithramycin (MTM), an aureolic acid-type polyketide antibiotic produced naturally by soil bacteria from the Streptomyces genus, exhibits anti-cancer activity by cross-linking GC-rich DNA and shutting down proto-oncogenes, particularly those triggered by Sp (specificity protein) transcription factors, e.g. Sp1 and Sp3. Overexpression of Sp1 has been observed in several cancers and has been linked to the control of cell growth, survival, and differentiation, all of which are important in cancer progression. MTM has been used clinically in the past to treat testicular cancer, Paget's bone disease, and hypercalcemia but has been severely limited by its poor bioavailability and toxic side effects such as hepatic, gastrointestinal, bone marrow, and renal toxicities. In order to address these issues, extensive combinatorial biosynthesis has been performed on the MTM pathway and has resulted in several novel analogues. The inventors have shown that the inactivation of the mtmW gene, which codes for the last enzyme in the MTM biosynthetic pathway, yields the most favorable of the new analogues, MTM SK (SK) and MTM SDK (SDK). See Albertini et al., Nucleic Acids Res.; 34(6):1721-1734 (2006) and Remsing et al., J Am Chem Soc. 125(19):5745-5753 (2003). Both SK and SDK exhibited higher anti-cancer activity than the native MTM, yet their short plasma retention time and low accumulation in tumors remained to be improved.

In an effort to aid with these shortcomings, nanoparticulate formulations for MTM analogues (e.g., SK and SDK), were prepared, for example, by using self-assembled or cross-linked polymer micelles from PEG-p(Asp-Hyd) block copolymers to which drugs were conjugated through acid-labile hydrazone bonds. SK and SDK were successfully loaded into polymer micelles and demonstrated a pH dependent release profile, accelerating drug release at pH below 7.4. By cross-linking the micelles, the size of the particles was increased without significantly affecting the drug loading. Both the self-assembled and cross-linked structures were able to protect the drugs in the biologically active form and efficiently kill the human non-small cell lung cancer cell line (A549) in vitro. MTM analogues entrapped in the cross-linked micelles interestingly improved the cytotoxicity as compared to the free drugs.

Accordingly, the present disclosure provides nanoparticulate formulations comprising polymer-based nanoparticle carriers and MTM or MTM analogues. The nanoparticulate formulations of the present disclosure improve or enhance the bioavailability, the plasma retention time and/or the intra-tumor accumulation of MTM or the MTM analogue. Each of the aspects of the present disclosure will be discussed in more detail below.

### DEFINITIONS

Specific ranges, values, and embodiments provided herein are for illustrative purposes and do not otherwise limit the scope of the disclosure. The term “about” is intended to encompass variations in physical values, and in amounts of ingredients and the like, owing to variations in weighing and other measurement techniques, purity of ingredients, and other factors, as would be known to the art worker. Such variations are often no more than about ±0.5%. The term “about” can thus indicate a variation of ±5 percent, or ±10 percent of the value specified; for example about 50 percent carries a variation from 45 to 55 percent; or the term can indicate ±1, 2, or 3 integers from the value specified.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configuration of the subject technology. A disclosure relating to an embodiment may apply to all embodiments, or one or more embodiments. An embodiment may provide one or more examples.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

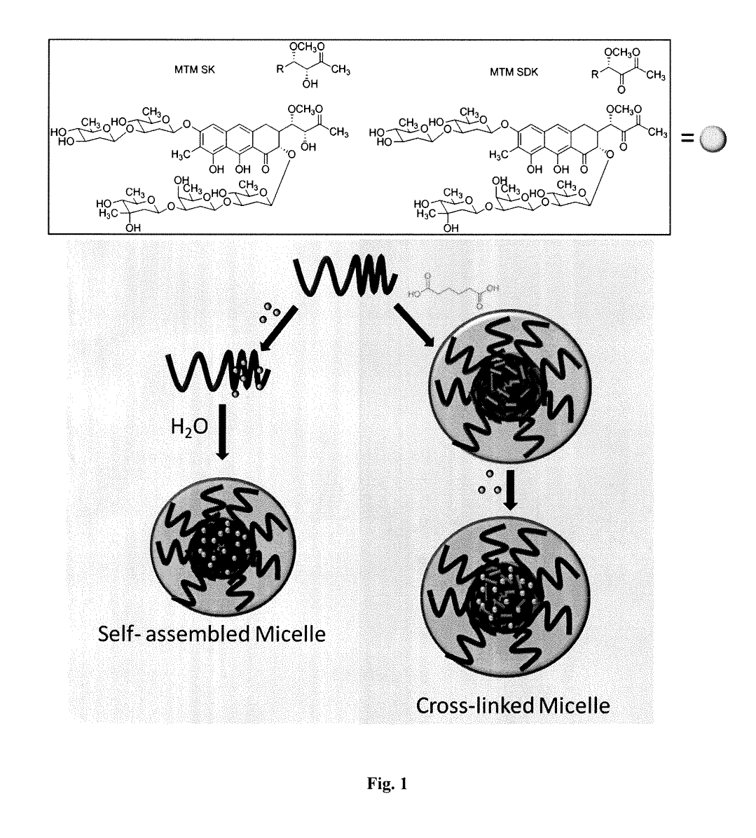

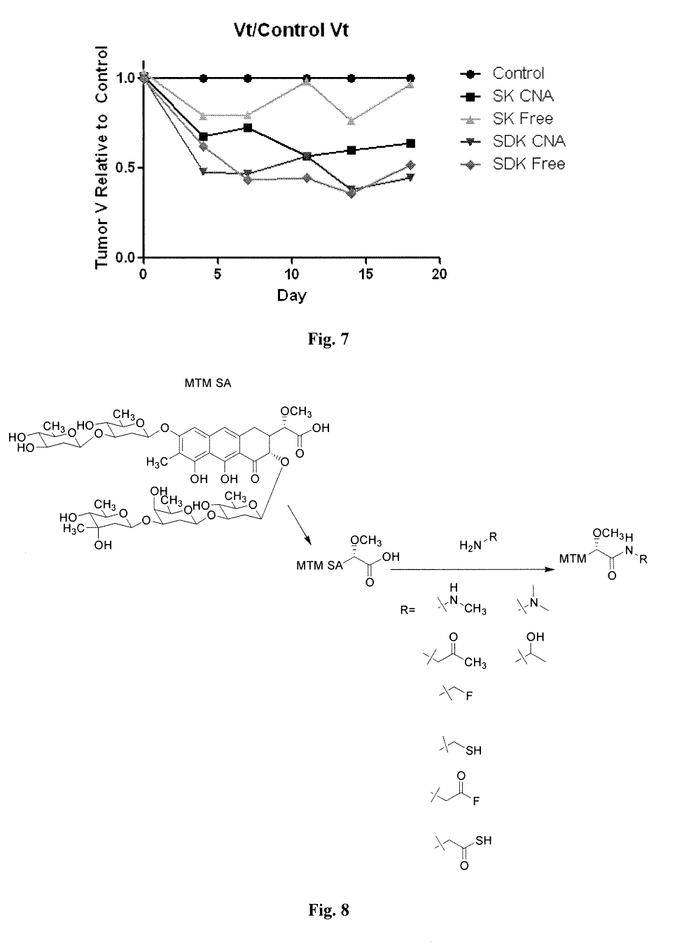

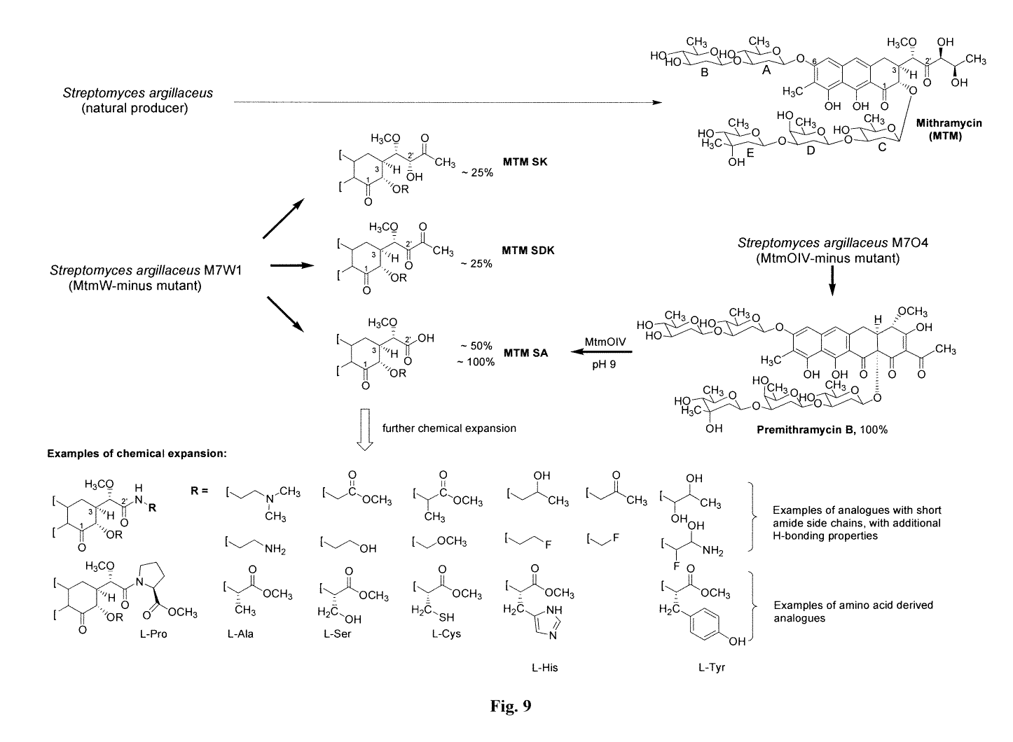

As used herein, an “MTM analogue” refers to any compound derived from mithramycin such as those depicted in FIG. 1, 8, or 9, i.e., SK (MTM with short side-chain and a ketone), SDK (MTM with short side-chain and diketone) or SA (MTM with short side-chain and carboxylic acid). Such derivatives may be further substituted or unsubstituted with an aliphatic or aromatic group. Preferably, an MTM analogue suitable for use in the nanoparticulate formulation of the present disclosure has a ketone group that can form a hydrazone bond to link or conjugate to the block co-polymer of the nanoparticulate formulation of the present disclosure. Possible MTM analogues suitable for use in the methods of the present disclosure also include MTM SA (as shown in FIG. 8) and all possible derivatives thereof which possess anti-cancer activity. MTM SA derivatives may be produced by substituting an MTM SA molecule with one or more different side-chains or functional groups. FIGS. 8 and 9 show, by way of example, preparation of various MTM analogues using an MTM SA molecule. The exemplary substitutable side-chains depicted in FIG. 8 are not to be construed as limiting the type or the number of side-chains or functional groups that can be substituted onto an MTM SA molecule to derive new MTM analogues. In an embodiment of the present disclosure, other therapeutic agents may be used in place of an MTM analogue, so long as such agent has a ketone group that can form a hydrazone bond with the co-polymers of the nanoparticulate formulation. Examples of such therapeutic agents are carboplatin, doxorubicin, docetaxel, idarubicin, mitocyim, mitoxantrone, paclitaxel, or geldanamycin and its derivatives.

The term “therapeutic agent” refers to biologically active agents, prodrugs, or drugs, including, for example, any organic or inorganic small molecule compound (e.g., a molecule with a molecular weight of less than about 1,000, or less and about 500), wherein said drug or agent has a ketone group and can be administered in vivo (in humans or animals) for the treatment of a disease, condition, or disorder. In one embodiment, the micelle cargo can be MTM or an MTM analogue such SK, SDK or SA or it can be a compound selected from the group consisting of carboplatin, doxorubicin, docetaxel, idarubicin, mitocyim, mitoxantrone, paclitaxel, and geldanamycin and its derivatives.

A “block copolymer” refers to a polymer with repeating units of one type adjacent to each other in a linear manner to form a block, which is linked, for example, through a covalent bond to a second block made up of repeating units of a second type, which are adjacent to one another in a linear manner to form a second block of the block copolymer. The block copolymers used in the formulations or methods of the present disclosure are further described in U.S. Patent Publication No. 2009/0232762 or in Lee et al., Biomacromolecules. Jul. 11, 2011; 12(7):2686-2696, each of which is hereby incorporated herein by reference.

According to one aspect of the subject technology, the nanoparticulate formulations of the present disclosure include block co-polymers of a polyamide linked to a biocompatible polymer such as, for example, polyethylene glycol (PEG). The polyamide portion of the block copolymer includes amino acid units derived from amino acid units with side chains that can have neutral, positive, or negative charges. The amino acid units of the first block can undergo a variety of reactions to provide multifunctional polyamides. Some side chain groups may remain as the initial unreacted side chains. Others can be converted to include functional groups such as, for example, hydrazide groups, which can facilitate the formation of pH-sensitive linkages such as, for example, acetal, orthoester, cis-aconityl group, hydrazone, imine, ester, Schiff base, dithioacetal, tert butyl ester, carbamate, thioester, or phosphoramidate. In some embodiments, more than 75% of the side chains of the amino acid units of the polyamide block will include pH-sensitive linkages. In some embodiments, more than 85% of amino acid side chains will include pH-sensitive linkages. In some embodiments, more than 90% of amino acid side chains will include pH-sensitive linkages. In some embodiments, more than 95% of amino acid side chains will include pH-sensitive linkages. Many of these linkages will link the amino acid units to a biocompatible polymer such as, for example, PEG groups by a condensation reaction, for example, between the N′ nitrogen and an aldehyde group of a PEG chain. The pH-sensitive linkages can further link the amino acid units to MTM or MTM analogues by, for example, a condensation reaction between the N′ nitrogen and a ketone group of MTM or MTM analogues.

“Hydrazone bond” is an exemplary pH-sensitive linkage and refers to the imine (N═C) bond of a hydrazone. In other words, the hydrazone bond refers to the bond formed between the N′ nitrogen of a hydrazine or hydrazide, and a carbonyl group of an aldehyde or ketone, which results in the formation of an N═C bond. The term hydrazone refers to an N-alkylidene derivative of a hydrocarbyl derivative of a hydrazine. The term hydrazide refers to a hydrazine that has an acyl substituent.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g. due to governmental regulations) for administration to a human. Except insofar as any conventional carrier is incompatible with the active ingredient, its use in the therapeutic or pharmaceutical compositions is contemplated.

As used herein, the term “nanoparticle” or “nanoparticulate” refers to polymer-based super- or macromolecular structures having dimensions in the 10-200 nm range which solubilize and stabilize their drug payload such as MTM or MTM analogues. In some embodiments, nanoparticles have dimensions in the 10-70 nm range, or in the 10-50 nm range, in 10 to 150 nm range or in 10 to 200 nm range. Nanoparticles used in the present disclosure include such nanoscale materials as a polymer-based micelle. In certain aspects, the nanoparticles can be conjugated to MTM or MTM analogues to provide structures with potential application for targeted delivery, controlled release, enhanced cellular uptake and intracellular trafficking in vitro and in vivo.

“Micelle” is an exemplary nanoparticulate super- or macromolecular structure that the block copolymers of the subject technology may form and refers to a supermolecular structure having a core-shell structure. Micelle formation is entropy driven and water molecules are typically excluded into the bulk phase. When above the critical micelle concentration (CMC), amphiphilic portions of the polymer employed aggregate into structured micelles. Polymeric micelles are typically spherical and can have nanoscopic dimensions in the range of about 10 to about 200 nm, typically in the 20-100 nm range. This is advantageous because circulating particles less than about 200 nm can avoid renal filtration. Additionally, delivery vehicles of less than about 150 nm are much more efficiently taken up by cells. Polymeric micelles have been shown to circulate in the blood for prolonged periods and are capable of targeted delivery of therapeutic agents, for example, nucleic acids or poorly water-soluble compounds. Upon disassociation, micelle monomers are typically <50,000 g/mol, permitting elimination by the kidneys. These properties allow for prolonged circulation with little or no buildup of micelle components in the liver that could lead to storage diseases. In an embodiment, the block copolymer carriers of the subject technology are cross-linked through their pH-sensitive linkages or self-assemble by electrostatic or hydrophobic forces to form micelles which will entrap MTM or MTM analogues and can increase stability and/or improved their in-vivo efficacy while reducing their toxicity.

“PEG” is an exemplary biocompatible polymer of the subject technology and refers to poly(ethylene glycol) and derivatives thereof. The molecular weight of the PEG chain can be about 500 to about 20,000. In certain embodiments, the PEG group can have a molecular weight of about 1,000 to about 20,000, about 2,000 to about 15,000, about 3,500 to about 12,000, or about 3,000 to about 9,000. In other embodiments, the PEG groups can have a molecular weight of about 4,000 or about 7,000. PEG groups can terminate in any variety of groups including hydroxyl, alkyl, alkoxy, aryl, arylalkyl, amino, and the like, referred to herein as PEG-capping groups. Other exemplary biocompatible polymers that can be used in the formulations of the subject technology include, but not limited to, chitosan (CS), polyethylenimine (PEI), poly(L-lactic acid) (PLLA), poly(D-lactic acid) (PDLA), poly(2-hydroxy ethyl methacrylate) (PHEMA), poly(ε-caprolactone) (PCL), poly(vinyl acetate) (PVAc), poly(ethylene oxide) (PEO), poly[(R)-3-hydroxybutyric acid)] (PHB), cellulose acetate (CA), poly(lactide-co-glycolide) (PLGA), poly(N-isopropylacrylamide) (PNIPAM), polyalkyleneglycol, polyalkyleneoxide, polyvinylpyrrolidone, polysaccharide, polyacrylamide, polymethacrylamide, polyvinylalcohol and derivatives thereof. Additional information about biocompatible polymers are provided in, for example, US20120003271, U.S. Pat. No. 7,226,616, each of which is hereby incorporated herein by reference. The biocompatible polymers of the subject technology can further be derivatized so long as such derivatives are still biocompatible and safe for administration to human. Routine methods for derivatizing polymers are known in the art.

The phrases “pharmaceutical” or “pharmacologically acceptable” refers to molecular entities and compositions that do not produce an adverse, allergic or other unwanted reaction when administered to an animal, such as a human, as appropriate. For animal (e.g., human) administration, it will be understood that preparations should meet sterility, pyrogenicity, general safety and purity standards as required by FDA Office of Biological Standards.

As used herein, the term “treatment” or “treating” refers to administration or application of a therapeutic agent to a subject or performance of a procedure or modality on a subject for the purpose of obtaining a therapeutic benefit of a disease or health-related condition. These benefits include cancer growth suppression, tumor regression, preventing cancer metastasis or recurrence and the like. For example, a treatment may include administration of a pharmaceutical formulation comprising MTM or an MTM analogue formulated in a pharmaceutically acceptable nanoparticulate formulation, whereby the nanoparticulate formulation solubilizes, stabilizes or improves the pharmacokinetics or pharmacodynamics of MTM or the MTM analogue in causing, inter alia, cancer growth suppression, tumor regression, preventing cancer metastasis or recurrence and the like.

A “subject” refers to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve a reduction in the size of a tumor, a reduction in the invasiveness of a tumor, reduction in the growth rate of the cancer, or prevention or reduction in metastasis. Treatment of cancer may also refer to prolonging survival of a subject with cancer.

A “disease” or “health-related condition” can be any pathological condition of a body part, an organ, or a system resulting from any cause, such as infection, genetic defect, and/or environmental stress. The cause may or may not be known. Specifically, a tissue or cell with the disease or condition may have an increased Sp1 or Sp3 (specificity protein 1 or 3) gene expression as compared with a normal tissue or cell. The formulations disclosed can be particularly used to treat such a cell or tissue because MTM or an MTM analogue is able to cross-link GC-rich DNA and shut down proto-oncogenes, particularly those triggered by Sp (specificity protein) transcription factors, e.g. Sp1 or Sp3, leading to selective tissue or cell killing.

A “hyperproliferative disease” includes diseases and conditions that are associated with any sort of abnormal cell growth or abnormal growth regulation, specifically a cancer.

As mentioned above MTM has been limited clinically by the severe negative side effects associated with the use of the drug. However, MTM has shown promise with respect to treating neurological diseases, glioblastomas, and other tumors in addition to showing the ability to inhibit the multi-drug resistance gene, MDR1 in which smaller, less toxic doses are required. Recently MTM was identified as the lead compound against the Friend leukemia virus integration 1 (EWS-FLI1) transcription factor. EWS-FLI1 is believed to be responsible for malignant transformation and progression of Ewing sarcoma family tumors and was widely regarded as undruggable because it is a transcription factor. A high-throughput screening of over 50,000 compounds identified MTM as the top candidate from the screen and greatly reduced and inhibited tumor volume in Ewing sarcoma family tumors in mouse xenograft models, and it is planned to reinstitute clinical trials for this application of MTM. Grohar et al., J. Natl. Cancer Inst. 2011; 103:962-978. Additionally, a combinational approach of MTM with betulinic acid to treat pancreatic cancer showed the ability to synergistically treat the cancer with a nontoxic dose of the drugs to produced less discernable side effects. As new treatments and uses for MTM continue to be uncovered the development and optimization of a compatible drug delivery system will be necessary.

The potential of MTM has been enhanced by the discovery of two new analogues and derivatives thereof produced through engineering of the MTM biosynthetic pathway. These analogues, SK and SDK, are a result of the inactivation of the mtmW gene and have shown improved activity compared to that of the native MTM. The biosynthesis and isolation of the SK and SDK analogues are pivotal as larger quantities of the drug will be required for testing and eventually clinical use. SK and SDK are produced alongside two other compounds, a demycarosyl-MTM SK and MTM SA (SA). The SA analogue (shown FIGS. 8 and 9) shows decreased anticancer activity and the demycarosyl-MTM SK shows activity similar to that of the native MTM, thus neither are the preferred products of the mutant strain. However, the SA analogue offers the unique opportunity to further functionalize the side chain by reacting the terminal carboxyl group. Initially a culture pH of 6.85 was used for the production of the drugs. These conditions allowed for good production of the SK but a large amount of SA and very little SDK were also being observed. The formation of SK, SDK or SA is a result of the spontaneous rearrangement of the immediate product MTM DK, resulting from the MtmOIV reaction of the biosynthetic pathway. It has been shown by in vitro work that the major product of the conversion of premithramycin B by MtmOIV can be shifted to SDK at a pH of 8.25. Gibson et al., J Am Chem Soc. Dec. 21, 2005; 127(50):17594-17595. In an effort to improve the production of SDK from the culture, the pH of the media was adjusted to 7.0. It was expected that the adjustment of the pH, although small, would still allow optimal growth of the M7W1 mutant strain and production of the MTM analogues, but that the production yield of SDK would be improved. Increasing the pH of the growth media to 7.0 confirmed the hypothesis and increased the production yield of SDK. Increasing the pH also did not appear to adversely affect the production of SK. This will be critical as the production of the analogues is scaled up to meet the clinical demands.

As the drive for more specific and efficient chemotherapeutic options continues, delivery systems will undoubtedly play a pivotal role in their development and advancement. To that end, issues such as low tumor accumulation, poor bioavailability and short plasma retention time for these novel MTM analogues still needed to be further explored. Drug delivery systems are expected to address these concerns by delivering MTM preferentially to tumors. Liposomal formulations have been previously tested, but the release of MTM or MTM analogues from liposomes proved difficult. Studies suggest that polymer micelles will offer an alternative platform for the delivery of MTM, not only delivering the drug to tumors but also controlling the drug release patterns efficiently. Based on the teachings of the present disclosure, it has been demonstrated that block copolymers offer a versatile platform in this direction.

In the present disclosure, MTM analogues (e.g., SK and SDK) were entrapped in polymeric micelles through an acid-labile hydrazone conjugation between drug molecules and micelle-forming block copolymers. Prepared MTM analogue nanoparticulate formulations combine the favorable characteristics of drug delivery systems with the ability to entrap drugs at high yields and to control the drug release at the area of interest.

SK and SDK have one and two ketone groups on the side chain, respectively. These ketone groups offer a prime location for the conjugation of the drugs to the PEG-p(Asp-Hyd) block copolymers through the hydrazone bond. Self-assembled micelles showed higher drug loading of SDK (44.6 wt %) than SK (35.4 wt %), suggesting that the second ketone group of the SDK side chain may facilitate drug loading rather than saturating the drug binding sites. The particle size of self-assembled micelles was 44% larger with SDK (12.19 nm) than SK (8.36 nm), which is consistent with the increased SDK loading in micelles. The average particle size of cross-linked micelles was 26.79 nm in the absence of drugs. Cross-linked micelles were 29.56 nm and 30.48 nm after entrapping SK and SDK, respectively. Even though the particle size was retained, cross-linked micelles entrapped 31.3 wt % SK and 42.7 wt % SDK, which were comparable to the amount of drugs that self-assembled micelles entrapped.

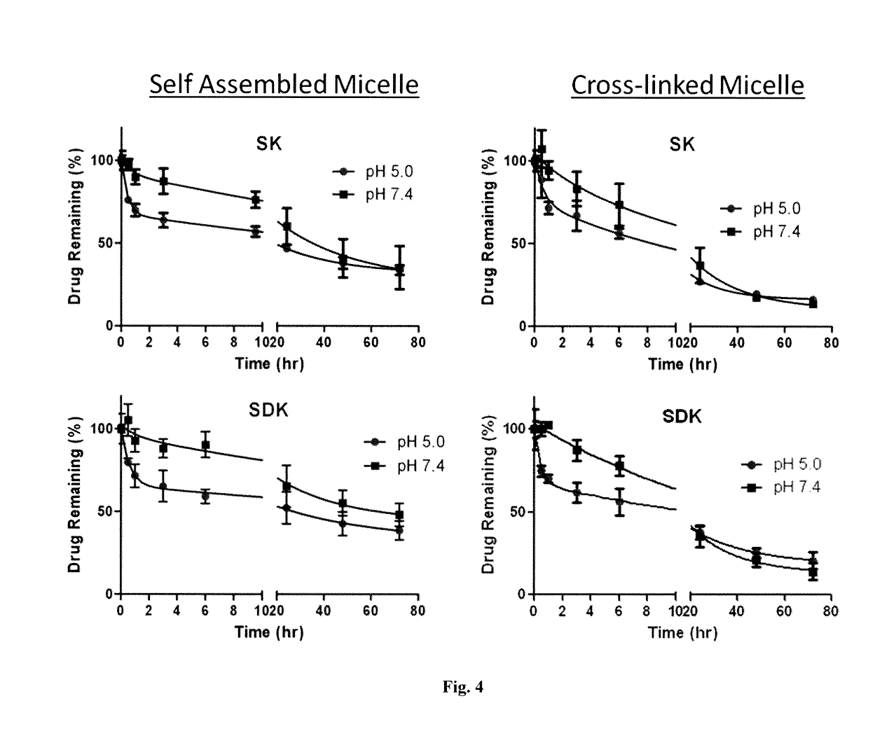

As an example, an acid labile hydrazone bond was used to conjugate the drugs to the block copolymers so the release of the drugs from the nano-formulations was investigated at both the physiological pH of 7.4 and an intracellular endosome or lysosome pH of 5.0. The effective drug release that was observed in all MTM analogue nanoparticulate formulations tested showed improvement over previous attempts of MTM delivery with liposomes. MTM analogues were released slowly from polymer micelles at pH 7.4 regardless of formulations. The drug release was accelerated at pH 5.0 (FIG. 4), which is advantageous to release drugs in acidic tumor tissues. After 72 hrs, however, the amount of drugs released was similar at both pH values. Thus, in one embodiment, the MTM analogue nanoparticulate formulations of the present disclosure can minimize drug leakage in the blood and maximize therapeutic efficacy by releasing drugs in tumor tissues rapidly.

As discussed in the Examples, the MTM analogue nanoparticulate formulations were shown to suppress cancer cell growth, indicating that MTM analogues were delivered in a biologically active form. Self-assembled micelles entrapping MTM analogues showed lower cytotoxic effects than free drugs, but surprisingly, cross-linked micelles enhanced drug efficacy up to 2.2 fold (Table 1).

TABLE 1
A summary of the characteristics and activity of the drug loaded polymer nanoparticles
and that of the free drugs for comparison.

| | SK | | | SDK | | |
|---|---|---|---|---|---|---|
| | | Self- | Cross- | | Self- | Cross- |
| Drug | | assembled | linked | | assembled | linked |
| Formulation | Free | Micelles | Micelles | Free | Micelles | Micelles |
| IC50 (nM) | 163 ± 92 | 262 ± 87 | 73 ± 15 | 93 ± 36 | 282 ± 131 | 75 ± 9 |
| Relative | 1.00 | 0.62 | 2.23 | 1.00 | 0.33 | 1.24 |
| efficacy to free | | | | | | |
| drug | | | | | | |

Without being bound by theory or mechanism of action, the results of the studies disclosed in the Examples below suggest that drug release in the early stage would not play a crucial role in determining cellular response to MTM analogues as all nanoparticulate formulations released a similar amount of drugs between 1-3 hrs. Drug release at pH 7.4 rather than 5.0 appeared to be the major factor that determines cytotoxicity. MTM analogues were effective to suppress cancer cell growth in order of cross-linked SK micelles, cross-linked SDK micelles, self-assembled SK micelles, and self-assembled SDK micelles, which is consistent with the drug release patterns at pH 7.4. Whereas all nanoparticulate formulations released drugs similarly at pH 5.0, which does not explain differential cytotoxicity observed in A549. Again, without being bound by theory or mechanism of action, it is more reasonable to assume that drugs released from micelles at pH 7.4 comprise the major drug population that involved in cell killing while intracellular drug release (pH 5.0) has less effect on overall cytotoxicity of MTM analogue nanoparticulate formulations.

For example, it was determined that the cross-linked micelles had a 12.5% cross-linking yield, which was high enough to tether block copolymer chains before and after entrapping drugs without causing agglomeration between particles. In spite of the high cross-linking yield, cross-linked micelles entrapped and released MTM analogues effectively. Importantly cross-linked micelles released drugs more quickly than self-assembled micelles, indicating that MTM analogues could be entrapped in the vicinity rather than at the center of the micelle core. The rapid drug release from cross-linked micelles can be also attributed to the rigid environment in the cross-linked core of the micelles. Cross-linking the core could allow for a more porous and less tightly packed core in the micelles so that drug molecules can maneuver more easily out of the core. To the contrary, self-assembled micelles have a flexible core that can continue to pull block copolymers to keep the particle stable as drugs are released. This may explain whydrug release from self-assembled micelles was slower than cross-linked micelles.

Cytotoxic effects of MTM analogue nanoparticulate formulations revealed an interesting phenomenon that can be used to design potent drug delivery systems. Unlike self-assembled micelles that decreased the toxicity of MTM analogues, cross-linked micelles showed a higher potency against A549 cancer cells than free drugs. The exact mechanism and reason for the improved efficacy is yet to be confirmed although the more stable cross-linked micelles could aid in the drug's persistence in the cells, allowing more drugs to interact with the therapeutic targets in the cell. In addition to the known mechanism of the MTM family of compounds in which they bind the GC rich DNA present in the Sp family of promoters, the expression of the gene encoding for a multi-drug resistance efflux pump, MDR1, has also been shown to be inhibited by MTM and some of it derivatives. This would make the MTM analogue nanoparticulate formulations excellent candidates for combinational therapy attacking hard-to-treat, multi-drug resistant (MDR) cancer cell lines.

As provided above, although the exact mechanism of the enhanced therapeutic efficacy of nanoparticulate formulations of mithramycin analogues is not yet known, without being bound by theory or mechanism of action, however, the intracellular uptake kinetics and distribution patterns of the drug seem to be determining factors. Accordingly, it is expected that all biocompatible macromolecules (1-50 kDa) and nanoscale materials (10-200 nanometers in diameter) could show effects similar to the micelle-forming copolymers of the subject technology. Thus, in certain embodiments, the subject technology relates to a pharmaceutical formulation comprising one or more active mithramycin analogues and a macromolecule. The mithramycin analogue includes, but not limited to, SK, SDK, SA or derivatives thereof, such as those shown in FIGS. 8 and 9. The macromolecule includes, but not limited to, natural, synthetic, semi-synthetic homopolymers and block copolymers (1-50 kDa) as well as their self-assembled or cross-linked supramolecular assemblies (5-200 nanometers in diameter). In an embodiment, relating to this aspect, one or more of the mithramycin analogues and the macromolecules are conjugated through covalent conjugation, metal chelation, or ionic interaction, which can be reversibly degradable in controlled manners. Covalent conjugations between mithramycin analogues and macromolecules include, but not limited to, imine, hydrazone, ester, disulfide, carbamate, and other degradable bonds. The method of controlling the degradation of the mithramycin and macromolecules includes, but not limited to, pH, heat, light, and enzymatic activity. In another related embodiment, the nanoscale supramolecular assemblies that are used as carriers or delivery vehicles include, but not limited to particles, micelles, vesicles, layers, membrane, sphere, tube, fiber, ribbon, sheet and the like. In another aspect, the subject technology relates to a method of delivering compositions comprising mithramycin analogues and macromolecules through oral, parenteral, pulmonary, transdermal, and other administration routes that are used by one in skill in the art. In another aspect, the subject technology relates to a method of treating a hyperproliferative disease or a quiescent malignant disease comprising administering to a subject in need thereof a therapeutically effective amount of the formulations described in this paragraph. In an exemplary embodiment relating to this aspect, SK or SDK or both formulated in a nanoparticle formulation comprising self-assembled or cross-linked poly(ethylene glycol)-poly(aspartate hydrazide)-based micelles.

### Nanoparticulate Formulation

In certain aspects, the present disclosure provides compositions and methods involving a polymer-based nanoparticulate formulation to improve the solubility, stability, bioavailability, pharmacokinetics and/or pharmacodynamics of MTM or MTM analogues for a better in vivo therapeutic activity. An MTM analogue, such as SK, SDK or SA is derived from MTM which is a natural product of soil bacteria of Streptomyces genus, which can be quickly metabolized and eliminated from the human body, and thus has limited bioavailability or therapeutic activity in vivo. The disclosed formulations, e.g., MTM-nanoparticles or MTM-analogue-nanoparticles, are designed to improve the solubility, stability, bioavailability as well as the pharmacokinetics and pharamcodynamics of MTM or the MTM analogues so that a better in vivo therapeutic activity can be achieved. For example, the nanoparticulate formulation may prolong the drug retention in blood circulation and increase the drug distribution to tumor tissues and uptake by cancer cells and thus enhance anticancer activity.

Exemplary polymer-based nanoparticles of the present disclosure include a pH-sensitive poly(ethylene glycol)-poly(aspartate hydrazide) block-copolymer in the form of self-assembled or cross-linked micelles as illustrated in FIG. 1. In certain nanoparticles of the present disclosure, PEG groups are attached through the formation of hydrazone bonds to hydrazide groups, forming a variable brush layer in the shell. At pH<7.4, acid labile PEG is released from the polymer, leaving behind hydrazide groups that enhance the escape of the drug payload (e.g., SK or SDK) form the core of the micelles. In one embodiment of the present disclosure, depending on reaction conditions and the desired properties of the polymers, the poly(ethylene glycol)-poly(aspartate hydrazide) block-copolymer has free hydrazide groups that can be used for conjugating MTM or the MTM analogues to the copolymer. For example, SK and SDK each have one and two ketone groups on the side chain, respectively. These ketone groups offer a prime location for the conjugation of the drugs to the PEG-p(Asp-Hyd) block copolymers through the hydrazone bond. The exemplary means for preparing the self-assembled or cross-linked micelles of the present disclosure are taught in the Examples provided below.

One aspect of the present technology concerns a novel MTM or MTM analogue nanoparticulate formulation for cancer treatment to overcome these problems, including the method and procedures to prepare the nanoparticles of proper size distribution and defined amount of MTM or MTM analogue suitable for administration in vivo. This MTM or MTM analogue nanoparticulate formulation has advantageous pharmacological properties including improved solubility, stability, bioavailability, pharmacokinetics, pharmacodynamics, effective anticancer activity, and favorable tolerability in animals.

In one aspect of the present disclosure, the subject technology provides a pharmaceutical composition comprising MTM or an MTM analogue (e.g., SK, SDK, SA or derivatives thereof), formulated in a pharmaceutically acceptable nanoparticulate formulation, whereby the nanoparticulate formulation increases or enhances the bioavailability, plasma retention time and/or intra-tumor accumulation of the MTM analogue.

The nanoparticulate formulation of the present disclosure comprises polymeric nanoparticles containing MTM or an MTM analogue or derivative such as, for example, those shown in FIGS. 8 and 9. In one embodiment, the nanoparticulate formulation of the present disclosure include (i) MTM or one or more MTM analogues or derivatives, as load; and (ii) block copolymer units of a polyamide and a biocompatible polymer as carrier. In a related embodiment, the polyamide includes amino acid units derived from amino acids with side chains that can have positive, neutral, or negative charges. In another related embodiment, the biocompatible polymer is selected from the group consisting of poly(ethylene glycol) (PEG), chitosan (CS), polyethylenimine (PEI), poly(L-lactic acid) (PLLA), poly(D-lactic acid) (PDLA), poly(2-hydroxy ethyl methacrylate) (PHEMA), poly(ε-caprolactone) (PCL), poly(vinyl acetate) (PVAc), poly(ethylene oxide) (PEO), poly[(R)-3-hydroxybutyric acid)] (PHB), cellulose acetate (CA), poly(lactide-co-glycolide) (PLGA), poly(N-isopropylacrylamide) (PNIPAM), polyalkyleneglycol, polyalkyleneoxide, polyvinylpyrrolidone, polysaccharide, polyacrylamide, polymethacrylamide, polyvinylalcohol and derivatives thereof. In another related embodiment, the copolymer units form nanoparticles in form of supermolecular structures selected from the group consisting of micelles, vesicles, layers, membrane, sphere, tube, fiber, ribbon and sheet. In a related embodiment, the block copolymer units self-assemble or crosslink to form a micelle with the polyamide forming the core and the biocompatible polymer forming the corona of the micelle. In another related embodiment, one or more of the amino acid units includes a pH-sensitive linkage in its side chain, wherein said pH-sensitive linkage is selected from the group consisting of acetal, orthoester, cis-aconityl group, hydrazone, imine, ester, Schiff base, dithioacetal, tert butyl ester, carbamate, thioester, or phosphoramidate. Exemplary pH-sensitive linkages or linkers are further described in, for example, U.S. Pat. No. 7,737,108, which is hereby incorporated herein by reference. In another related embodiment, the amino acid units includes aspartic acid or glutamic acid or both. In another related embodiment, the MTM or one or more of the MTM analogues is conjugated to one or more of the amino acid units via the pH-sensitive linkage. In another related embodiment, the pH-sensitive linkage is stable at a pH between about 7 and 8 and is hydrolyzed at a pH less than about 7 to release the MTM or one or more of the MTM analogues. In another related embodiment, the block copolymer is poly(ethylene glycol)-poly(aspartate hydrazide) copolymer. In another related embodiment, the formulation is freeze dried to form a freeze-dried formulation.

In another embodiment, the nanoparticulate formulations of the subject technology improve the solubility, stability, bioavailability, anticancer activity, anticancer efficacy, pharmacokinetics, pharmacodynamics, or tolerability of their drug payloads (i.e., MTM or MTM analogues) by at least 10% over the free drug. In another embodiment, the nanoparticulate formulations of the subject technology improve the solubility, stability, bioavailability, anticancer activity, anticancer efficacy, pharmacokinetics, pharmacodynamics, or tolerability of their drug payloads (i.e., MTM or MTM analogues) by at least 1.1 fold over the free drug. In yet another embodiment, the nanoparticulate formulations of the subject technology improve the solubility, stability, bioavailability, anticancer activity, anticancer efficacy, pharmacokinetics, pharmacodynamics, or tolerability of their drug payloads (i.e., MTM or MTM analogues) by at least 1.1 fold over the free drug.

Pharmacokinetics, sometimes described as what the body does to a drug, refers to the movement of drug into, through, and out of the body the time course of its absorption, bioavailability, distribution, metabolism, and excretion. Drug pharmacokinetics determines the onset, duration, and intensity of a drug's effect. Formulas relating these processes summarize the pharmacokinetic behavior of most drugs. See Table 2 below. Based on these information and those provided herein, the Examples section, a person of ordinary skill in the art can readily assess the improvements in solubility, stability, bioavailability, anticancer activity, anticancer efficacy and pharmacokinetics of the formulations of the subject technology.

TABLE 2
Formulas Defining Basic Pharmacokinetic Parameters

| Category | Parameter | Formula |
|---|---|---|
| Absorption | Absorption rate constant | Rate of drug absorption ÷ |
| | | amount of drug remaining to |
| | | be absorbed |
| | Bioavailability | Amount of drug absorbed ÷ |
| | | drug dose |
| Distribution | Apparent volume of | Amount of drug in body ÷ |
| | distribution | plasma drug concentration |
| | Unbound fraction | Plasma concentration of |
| | | unbound drug ÷ plasma drug |
| | | concentration |
| Elimination | Rate of elimination | Renal excretion + extrarenal |
| | | (usually metabolic) |
| | | elimination |
| | Clearance | Rate of drug elimination ÷ |
| | | plasma drug concentration |
| | Renal clearance | Rate of renal excretion of drug ÷ |
| | | plasma drug concentration |
| | Metabolic clearance | Rate of drug metabolism ÷ |
| | | plasma drug concentration |
| | Fraction excreted | Rate of renal excretion of drug ÷ |
| | unchanged | rate of drug elimination |
| | Elimination rate constant | Rate of drug elimination ÷ |
| | | amount of drug in body |
| | | Clearance ÷ volume of |
| | | distribution |
| | Biologic half-life | 0.693 ÷ elimination rate |
| | | constant |

Pharmacokinetics of a drug depends on patient-related factors as well as on the drug's chemical properties. Some patient-related factors (eg, genetic makeup, sex, age) can be used to predict pharmacologic response of populations. For example, the half-life of some drugs, especially those that require both metabolism and excretion, may be remarkably long in the elderly. In fact, physiologic changes with aging affect many aspects of pharmacokinetics; thus to assess improvements in pharmacokinetics of the formulations of the subject technology, it is recommended that the test subjects and the control subjects be of the same age. Other factors are related to individual physiology. The effects of some individual factors (eg, renal failure, obesity, hepatic failure, dehydration) can be reasonably predicted, but other factors are idiosyncratic and thus have unpredictable effects. Because of individual differences, drug administration must be based on each patient's needs—traditionally, by empirically adjusting dosage until the therapeutic objective is met. Routine methods for assessing pharmacokinetics of the drugs that help prescribers adjust dosage more accurately and rapidly are known in the art; see for example, U.S. Pat. Nos. 8,155,737; 8,133,892; 7,722,595; 8,052,617, each of which is hereby incorporated herein by reference.

The nanoparticulate formulation of the present disclosure can have a particle diameter of less than 200 nm, about 10 nm to about 50 nm, about 15 to 70 nm, about 10 to about 50 nm, about 15 to about 100 nm, about 15 to about 150 nm, about 10 to about 200 nm or any range derivable therein. The particle diameter can be a mean or average diameter. In an exemplary embodiment, the nanoparticulate formulations of the present disclosure has an average particle size of 8.36±3.21 and 12.19±2.77 nm for the SK and SDK self-assembled micelles and 29.56±4.67 nm and 30.48±7.00 nm for the SK and SDK cross-linked micelles, respectively.

Exemplary co-polymers used in the nanoparticulate formulation of the present disclosure include, but are not limited to, poly(ethylene glycol)-poly(aspartate hydrazide) co-polymer. Other copolymers that can be used in nanoparticulate formulations of the subject technology include block copolymer units of a polyamide and a biocompatible polymer. In an embodiment, the polyamide includes amino acid units derived from amino acids with side chains that can have positive, neutral, or negative charges. In another embodiment, the biocompatible polymer is selected from the group consisting of poly(ethylene glycol) (PEG), chitosan (CS), polyethylenimine (PEI), poly(L-lactic acid) (PLLA), poly(D-lactic acid) (PDLA), poly(2-hydroxy ethyl methacrylate) (PHEMA), poly(ε-caprolactone) (PCL), poly(vinyl acetate) (PVAc), poly(ethylene oxide) (PEO), poly[(R)-3-hydroxybutyric acid)] (PHB), cellulose acetate (CA), poly(lactide-co-glycolide) (PLGA), poly(N-isopropylacrylamide) (PNIPAM), polyalkyleneglycol, polyalkyleneoxide, polyvinylpyrrolidone, polysaccharide, polyacrylamide, polymethacrylamide, polyvinylalcohol and derivatives thereof. In another embodiment, one or more of the amino acid units of the polyamide block includes a pH-sensitive linkage in its side chain, wherein said pH-sensitive linkage is selected from the group consisting of acetal, orthoester, cis-aconityl group, hydrazone, imine, ester, Schiff base, dithioacetal, tert butyl ester, carbamate, thioester, or phosphoramidate. In another embodiment the pH-sensitive linkage is used to cross-link the block copolymer units of a polyamide and a biocompatible polymer together. In another embodiment the pH-sensitive linkage is used to link and entrap MTM or MTM analogues in the nanoparticle carriers of the subject technology. In another embodiment, the pH-sensitive linkage is stable at a pH between about 7 and 8 and is hydrolyzed at a pH less than about 7 to release the MTM or one or more of the MTM analogues. In another embodiment, the pH-sensitive linkage hydrolyzes within 30-120 minutes at pHs between 5.0-6.5.

Other exemplary co-polymers that can be used in the nanoparticulate formulation of the present disclosure are those described in U.S. Patent Publication No. 2009/0232762 and Lee et al. Biomacromolecules. Jul. 11, 2011; 12(7):2686-2696, including a polyamide block polymer comprising at least one block of poly(aspartic acid) units; wherein at least one side chain of an aspartic acid unit is covalently linked to a poly(ethylene glycol) chain through a hydrazide moiety, and wherein the hydrazide moiety is linked to the poly(ethylene glycol) (PEG) chain at the N nitrogen of the hydrazide through a hydrazone bond. The co-polymers of the present disclosure are pH-sensitive. At a pH of less than approximately 7.4, the acid PEG moieties (forming the shell of the micelles) can be released from the polymer, leaving behind hydrazide groups that can enhance the escape of MTM or MTM analogues from the nanoparticles of the subject technology.

The nanoparticulate formulation of the present disclosure can be a liquid formulation or a solid formulation, such as a powder. Particularly, the composition may be dehydrated or lyophilized for long term storage with improved stability. Alternatively, the composition may be present in a substantially aqueous solution. The composition may be rehydrated or re-suspended in a solution or liquid from the previously lyophilized composition.

In an exemplary embodiment, the present disclosure concerns a pharmaceutical composition including MTM or an MTM SK, SDK or SA analogue or any combination of said molecules formulated in a nanoparticulate formulation comprising self-assembled or cross-linked poly(ethylene glycol)-poly(aspartate hydrazide)-based micelles, whereby the nanoparticulate formulation solubilizes or stabilizes said MTM or said MTM SK, SDK or SA analogue or said any combination of said molecules, or improves the bioavailability, therapeutic activity, pharmacokinetics or pharmacodynamics of said MTM or said MTM SK, SDK or SA analogue or said any combination of said molecules in-vivo.

In another embodiment, the nanoparticulate formulations of the present disclosure include polymer-based nanoparticles containing MTM or an MTM analogue. In a related embodiment, the nanoparticulate formulations of the present disclosure comprise poly(ethylene glycol)-poly(aspartate hydrazide) copolymers. In another embodiment, the polymeric nanoparticles of the present disclosure are self-assembled in a core-shell structure to protect their drug payloads (i.e., MTM or MTM analogues) from precipitation. In another related embodiment, the polymeric nanoparticles of the present disclosure are cross-linked in the core to which their drug payloads (i.e., MTM or MTM analogues) conjugate through pH-sensitive linkages such as, for example, hydrazone bonds.

In another embodiment, the nanoparticles of the subject technology improve the solubility, stability, bioavailability, anticancer activity, anticancer efficacy, pharmacokinetics, pharmacodynamics, or tolerability of their drug payloads (i.e., MTM or MTM analogues) by at least 10% over the free drug. In another embodiment, the nanoparticles of the subject technology improve the solubility, stability, bioavailability, anticancer activity, anticancer efficacy, pharmacokinetics, pharmacodynamics, or tolerability of their drug payloads (i.e., MTM or MTM analogues) by at least 1.2 fold over the free drug. In yet another embodiment, the nanoparticles of the subject technology improve the solubility, stability, bioavailability, anticancer activity, anticancer efficacy, pharmacokinetics, pharmacodynamics, or tolerability of their drug payloads (i.e., MTM or MTM analogues) by at least 1.1 fold over the free drug.

The nanoparticulate formulation of the present disclosure can further comprise an additional therapeutic agent to exert an additive or synergistic benefit, such as a chemotherapeutic agent as discussed below.

### Polymeric Nanoparticles as Drug Delivery Vehicles

An effective drug delivery system ensures that drug molecules are delivered to disease lesions at the right amount and timing by using drug carriers. Nanoparticles are widely used as drug carriers because large molecules (>50 kDa) can avoid renal clearance and circulate in the body for prolonged time in comparison to small molecules. Tumors are characterized with leaky blood vessels and immature lymphatic drainage, which allow nanoparticles (10-200 nm) to accumulate in tumor tissues. Nanoparticles take advantage of both prolonged plasma retention time and preferential tumor accumulation to deliver drug payloads to tumors preferentially. Liposomes, dendrimers, and polymer micelles are exemplary nanoparticles used widely for tumor-preferential delivery of therapeutic agents. Among these formulations, polymer micelles offer a versatile platform that can be prepared through self-assembling of block copolymers in aqueous solutions. Self-assembled polymer micelles possess a core compartment enveloped by a hydrophilic shell. The core-shell structure protects drug payloads from precipitation, protein adsorption and enzymatic degradation in the body.

Thus, in certain aspects of the subject technology, the polymeric nanoparticles can be in the form of micelles, either cross-linked or self-assembled micelles. In one embodiment, the polymer micelles of the present disclosure can dissociate as the core becomes unstable under conditions accompanied by dilution, drug release, or polymer degradation. Polymer micelles can be stabilized by cross-linking in the core or shell to avoid premature micelle dissociation during tumor-preferential drug delivery.

In addition to tumor-preferential drug delivery, controlled drug release is another important factor in maximizing therapeutic efficacy of drug payloads. Drug binding linkers are normally designed to degrade in response to pH, enzymatic activity, light, or heat. The hydrazone bond, involved in many biomolecular events, is stable at physiological pH (7.4), yet is cleaved in acidic conditions (pH<7.0) in a pH-dependent manner. Such a unique degradation pattern has been employed herein to design pH-controlled drug delivery systems that can trigger drug release in intracellular lysosomal compartments (pH 5.0), following the cellular uptake of drug carriers. Tumor tissues are also acidic (pH 6.5-7.0) due to the inefficient glucose consumption that massively produces lactic acids. Low pH in tumors is also attributed to high flux at the glyceraldehydes-3-phosphate dehydrogenase step of glycolysis. These two factors contribute to tumor acidosis.

In one embodiment of the present disclosure, MTM or MTM analogues are conjugated to the polymer micelles through acid-sensitive or pH-sensitive hydrazone linkers. The hydrazone is a chemical bond between a ketone and hydrazide groups, and thus, drug molecules containing ketone groups are advantageous to the design of pH-sensitive drug delivery systems.

FIGS. 1, 8 and 9 show the chemical structures of MTM analogues (SK and SDK) and two nanoparticulate formulations (self-assembled and cross-linked polymer micelles) used in this disclosure. SK and SDK possess one and two ketone groups, respectively, in their 3-side chains, plus an additional keto group in the tricyclic ring system. At least one of the ketone groups is subsequently bound to hydrazide groups of poly(ethylene glycol)-poly(aspartate hydrazide) [PEG-p(Asp-Hyd)] block copolymers to prepare drug-conjugated polymer micelles. The acid-labile hydrazone bonds between the block copolymers and MTM analogues are used to accelerate drug release in acidic conditions corresponding to tumor tissues in vivo. In the Examples that follow, self-assembled and cross-linked micelles were compared and the influence of micelle stability on drug release patterns was studied. A549 cell lines (human non-small cell lung cancer) were used to test biological activity of nanoparticulate MTM formulations.

Thus, in a certain aspects, the subject technology includes a delivery system that can be used to deliver biologically active agents or formulations or pharmaceutical compositions to a target. In one embodiment of this aspect of the subject technology, the disclosure includes the delivery of biologically active agents by loading them into nanoparticles comprising an amphiphilic core and a hydrophilic outer surface, thus improving their delivery in aqueous media, such as blood and body fluids. In other aspects, the disclosure includes the delivery of biologically active agents while reducing their toxicity profile. The disclosure also includes a method for reducing aggregation of the micelle delivery vesicles of the disclosure. As such, it provides for better biodistribution of biologically active agents resulting in decreased toxicity and/or improved therapeutic efficacy.

Another aspect of the subject technology includes a method of delivering biologically active agents to treat a disease, condition, or disorder in a subject in need thereof comprising administering an effect amount of an agent-loaded micelle to a subject. In one embodiment, the disease, condition or disorder is cancer or drug resistant cancers, infectious disease or an autoimmune disease.

One useful aspect of micelle carriers is that they can be employed for the delivery of therapeutic agents without chemically modifying the agent. The structure of the polymers described herein can be tailored in order to enhance the properties of the micelles for therapeutic agent delivery. Such tailoring includes varying the number of, for example, hydrazide groups.

The exemplary micelles disclosed herein allow for the biocompatible polymer, e.g., PEG side-groups of the polymers to be concentrated at the outer portions of the micelles, referred to as the micelle corona. The micelle corona is therefore hydrophilic and allows for its incorporation into cells.

One advantage of nanoparticulate compositions (e.g., those including micelles) includes their ease of storage and delivery. Such compositions can be lyophilized and reconstituted before intravenous administration. This allows for a lower risk of agent precipitation, which can in some cases lead to embolism formation. Nanoparticulate compositions are capable of long blood circulation, low mononuclear phagocyte uptake, and low levels of renal excretion. Nanoparticulate compositions also have enhanced permeability and retention (EPR) to increase the likelihood of their encapsulated therapeutics reaching their targets, for example, tumors.

Tumors typically have high vascular density, as well as defective vasculature. Accordingly, high extravasation occurs and there may be impaired lymphatic clearance. The endocytosis and subsequent nanoparticle (e.g., micelle) disaggregation allows for the release of the encapsulated agent its delivery into the cell.

In various embodiments, the unloaded or empty nanoparticles can be prepared. In other embodiments, the resultant nanoparticles can have average diameters of less than about 200 nm, or less than about 100 nm. In another embodiment, the nanoparticles can have an average diameter of between about 10 nm and about 50 nm.

The small size of polymeric nanoparticles that have PEG coronas can help the micelle carrier to stay unrecognized, as self, in a biological system. Other advantages associated with nanoscopic dimensions of polymeric nanoparticles include the ease of sterilization via filtration and safety of administration. The core of the nanoparticles can take up, protect and retain biologically active agents, leading to improved solubility and stability of the agents in vivo, their controlled release, and overall reduced toxicity and attenuated pharmacokinetic interaction with other treatment agents.

Another aspect of the subject technology includes a method of delivering MTM or MTM analogues to treat a disease, condition, or disorder in a subject in need thereof comprising administering an effective amount of MTM or an MTM analogue-loaded micelle to the subject. In one embodiment, the disease, condition or disorder is cancer or drug resistant cancer.

In another embodiment, the present disclosure related to a method of delivering MTM or an MTM SK, SDK or SA analogue or any combination of said molecules to a subject in need thereof, including administering to the subject a therapeutically effective amount of a nanoparticulate formulation comprising self-assembled or cross-linked poly(ethylene glycol)-poly(aspartate hydrazide)-based micelles containing said MTM or said MTM SK, SDK or SA analogue or said any combination of said molecules.

The dosage of the nanoparticles of the disclosure can vary depending on many factors such as the pharmacodynamic properties of the nanoparticles, the pharmacodynamic properties of MTM or the MTM analogue used, the rate of release of the agent from the nanoparticles, the mode of administration, the age, health and weight of the recipient, the nature and extent of the symptoms, the frequency of the treatment and the type of concurrent treatment, if any, and the clearance rate of the agent and/or nanoparticle in the subject to be treated.

One of skill in the art can determine the appropriate dosage based on the above factors. The nanoparticles may be administered initially in a suitable dosage that may be adjusted as required, depending on the clinical response. For ex vivo treatment of cells over a short period, for example for 30 minutes to 1 hour or longer, higher doses of nanoparticles may be used than for long term in vivo therapy.

The loaded nanoparticles can be used alone or in combination with other agents that treat the same and/or another condition, disease or disorder. In another embodiment, where either or both the nanoparticle or biologically active agent is labeled with a detectable label, one can conduct in vivo or in vitro studies for determining optimal dose ranges, drug loading concentrations and size of nanoparticles and targeted drug delivery for a variety of diseases.

### Administration of Nanoparticulate Formulation

The polymer-based nanoparticles of the present disclosure can be suitably formulated into pharmaceutical compositions for administration to human subjects in a biologically compatible form suitable for administration in vivo. Accordingly, in certain embodiments, a pharmaceutical composition is provided that includes nanoparticles as described herein, in admixture with a suitable diluent or carrier.

The compositions containing nanoparticles can be prepared by known methods for the preparation of pharmaceutically acceptable compositions that can be administered to subjects, such that an effective quantity of the therapeutic agent within the nanoparticles is combined in a mixture with a pharmaceutically acceptable vehicle, such as enteric coating. For example, the nanoparticles of the formulations of the subject technology can have enteric coating to protect them from acidity of the gastric fluid if such formulations are to administered orally. Suitable vehicles are described, for example, in Remington's Pharmaceutical Sciences (2003, 20.sup.th Ed.), in The United States Pharmacopeia: The National Formulary (USP 24 NF19) published in 1999, and in the Handbook of Pharmaceutical Additives (compiled by Michael and Irene Ash, Gower Publishing Limited, Aldershot, England (1995)). On this basis, the compositions include, albeit not exclusively, solutions of the nanoparticles in association with one or more pharmaceutically acceptable vehicles or diluents, and contained in buffered solutions with a suitable pH and iso-osmotic with the physiological fluids. In this regard, reference can be made to U.S. Pat. No. 5,843,456 (Paoletti and Maki). In one embodiment, the pharmaceutical compositions can be used to enhance biodistribution and drug delivery of therapeutic agents, such as drugs.

In an embodiment, the composition of this disclosure enables sustained, continuous delivery of MTM or MTM analogues such as SK or SDK to tissues adjacent to or distant from an administration site. The biologically-active agent is capable of providing a local or systemic biological, physiological or therapeutic effect. For example, MTM or MTM analogue may act to kill cancer cells, or cancer stem cells or to control or suppress tumor growth or metastasis, among other functions.

In one embodiment, the MTM or MTM analogue (e.g., SK or SDK) nanoparticles formulations of the present disclosure are administered in an amount effective to provide the desired level of biological, physiological, pharmacological and/or therapeutic effect. The MTM or MTM analogue nanoparticulate formulations may stimulate or inhibit a biological or physiological activity. The lower limit of the amount of MTM or the MTM analogue will depend on its activity and the period of time desired for treatment. In another embodiment, MTM or the MTM analogue of the formulations of the present disclosure may be gradually released, in a controlled release manner, by dissolution of the nanoparticles (e.g., polymer-based micelles).

The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical formulations include, for example, at least about 0.1% of an active compound, such as MTM, SK, SDK, SA or derivatives thereof. In other embodiments, the active compound may comprise between about 1% to about 75% of the weight of the unit dosage, or between about 5% to about 50% by weight of the unit dosage, for example, and any specific percentage in between these ranges. In other non-limiting examples, a dose may also comprise from about 0.01 microgram/kg/body weight, about 50 microgram/kg/body weight, about 100 microgram/kg/body weight, about 500 microgram/kg/body weight, about 1 milligram/kg/body weight, about 5 milligram/kg/body weight, about 10 milligram/kg/body weight, about 30 milligram/kg/body weight, about 40 milligram/kg/body weight, about 50 milligram/kg/body weight, about 100 milligram/kg/body weight, or more per administration, and any range or specific amount derivable therein. In non-limiting examples of a derivable range from the numbers listed herein, a range of about 5 microgram/kg/body weight to about 5 milligram/kg/body weight, about 50 microgram/kg/body weight to about 50 milligram/kg/body weight, etc., can be administered.

For a safe and effective dosage, the formulations can be administered at a dose of about 0.01 to about 500 mg/m² (body surface)/day, about 0.01 to about 300 mg/m²/day, 0.01 to about 200 mg/m²/day, about 1 to about 200 mg/m²/day about 10 to about 100 mg/m²/day, about 25 to about 100 mg/m²/day or any range derivable therein to a subject such as a human. In certain aspects, the composition may be administered at a dose of about 0.01 to about 200 mg/kg body weight, about 0.01 to about 100 mg/kg body weight, 1 to about 50 mg/kg body weight, about 1 to about 20 mg/kg body weight, about 3 to about 10 mg/kg body weight, about 3 to about 6 mg/kg body weight or any range derivable therein to a subject such as a human. In some embodiments, a nanoparticulate formulation of the subject technology may be administered in a dose of 1, 2, 3, 4, 5, 6, 7, 8, 9, 10, 15, 20, 25, 30, 40, 50, 60, 70, 80, 90, 100 mg or more per day. Each dose may be in a volume of 1, 10, 50, 100, 200, 500, 1000 or more μl or ml.

In some embodiments, a the pharmaceutical formulation of the subject technology includes MTM or an MTM analogue in an amount effective to result in a serum concentration of the MTM or the MTM analogue in the mammal in a range of from 1 mM to 1 M.

Examples of Serum and Systemic Circulation Concentrations of MTM or MTM analogues: Serum and systemic circulation concentrations of MTM or MTM analogues effective to result in the treatment, prevention or regression of tumor or cancer may vary depending on a number of factors. Influential variables can include, for example, various chemical properties of one MTM analogue, as compared to another. For example different MTM analogues can differ in pKa, solubility, molecular weight, etc., and these properties of a particular MTM analogue may affect how a patient metabolizes the analogue, how much of the analogue enters and remains in the systemic circulation of a mammal, and how effectively the analogue treats, prevents or causes regression of the tumor or cancer.

Accordingly, in some embodiments of the subject technology, a serum or a systemic circulation concentration of an MTM analogue effective to result in the treatment, prevention, or regression of cancer may be in a range of from, for instance, about 1 μM to about 10 μM, about 5 μM to about 10 μM, about 10 μM to about 20 μM, about 20 μM to about 30 μM, about 30 μM to about 40 μM, about 40 μM to about 50 μM, about 50 μM to about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μMto about 300 μM, about 100 μM to about 550 about 150 μM to about 500 about 200 μM to about 450 μM, about 250 μM to about 400 μM, about 300 μM to about 350 μM, about 500 to about 600 about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

Examples of MTM analogue Doses: In some embodiments, an MTM analogue dose effective to result in the treatment, prevention, or regression of cancer may be, in weight of administered MTM analogue per kilogram of mammal body weight per day (mg/kg/day), in a range of from, for instance, about 0.01 mg/kg/day to about 1 mg/kg/day, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

The therapeutic formulations of the present disclosure are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

An effective amount of the therapeutic formulation is determined based on the intended goal. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are peculiar to each individual. Factors affecting the dose include the physical and clinical state of the patient, the route of administration, the intended goal of treatment (e.g. alleviation of symptoms versus cure) and the potency, stability and toxicity of the particular therapeutic substance.

### Route of Administration

In accordance with the methods of the disclosure, the described nanoparticles, may be administered to a subject in a variety of forms depending on the selected route of administration, as will be understood by those skilled in the art. The nanoparticles of the disclosure may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, intratumoral, transepithelial, nasal, intrapulmonary, intrathecal, rectal and topical modes of administration. Parenteral administration may be by continuous infusion over a selected period of time.

A nanoparticle may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the nanoparticle of the disclosure may be incorporated with excipient and used in the form of ingestible tablets, buccal tablets, troches, capsules, elixirs, suspensions, syrups, wafers, and the like. A nanoparticle may also be administered parenterally.

Solutions of a nanoparticle can be prepared in water suitably mixed with suitable excipients. Under ordinary conditions of storage and use, these preparations can contain a preservative to prevent the growth of microorganisms.

The pharmaceutical forms suitable for injectable use include sterile aqueous solutions or dispersion and sterile powders for the extemporaneous preparation of sterile injectable solutions or dispersions. The form should be sterile and should be fluid to the extent it makes injection possible.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compositions described herein can be administered to an animal alone or in combination with pharmaceutically acceptable carriers, as noted above, the proportion of which is determined by the solubility and chemical nature of the compound, chosen route of administration and standard pharmaceutical practice. In an embodiment, the pharmaceutical compositions are administered in a convenient manner such as by direct application to the tumor, e.g. by injection (intratumorally, subcutaneous, intravenous, etc.). Depending on the route of administration (e.g. injection, oral, or inhalation, etc.), the pharmaceutical compositions or nanoparticles or biologically active agents in the nanoparticles of the disclosure may be coated in a material to protect the nanoparticles or agents from the action of enzymes, acids, and other natural conditions that may inactivate certain properties of the composition or its encapsulated agent.

Thus, in certain embodiments, the formulations of the present disclosure can be administered to a subject in need thereof intravenously, intradermally, intraarterially, intraperitoneally, intralesionally, intracranially, intraarticularly, intraprostaticaly, intrapleurally, intratracheally, intravitreally, intravaginally, intrarectally, intratumorally, intramuscularly, intraperitoneally, subcutaneously, subconjunctival, intravesicularlly, mucosally, intrapericardially, intraumbilically, intraocularally, intrathecally, locally, by injection, by infusion, by continuous infusion, by localized perfusion bathing target cells directly, via a catheter, or via a lavage. For example, the composition can be administered by injection or infusion.

### Method of Treatment

In a further aspect of the disclosure, the present disclosure provides a method of treating a hyperproliferative disease or a quiescent malignant disease comprising administering a therapeutically effective amount of the nanoparticle composition described above to a subject in need of such treatment. The composition used in this method may be previously dehydrated, lyophilized or in some other aspects, an aqueous solution or liquid formulation of previously lyophilized or dehydrated composition, an effective amount of which are administered to the subject. The present disclosure also provides, in certain aspects, previously lyophilized or dried composition after being stored at 4° C. for at least 1 week, for at least 3 weeks, for up to 4 weeks, or any period derivable therein, for treating the disease with retained activity after resuspension or rehydration.

Hyperproliferative Diseases: In certain embodiments of the disclosure, a therapeutically effective amount of the pharmaceutical composition comprising an MTM analogue formulated in a pharmaceutically acceptable nanoparticulate formulation may be used to treat a diseases and/or condition in a subject. In some embodiments of the disclosure, the methods include identifying a patient in need of treatment. A patient may be identified, for example, based on taking a patient history, based on findings on clinical examination, based on health screenings, or by self-referral.

The subject technology can find application in the treatment of any disease for which delivery of a therapeutic MTM or MTM analogue to a cell or tissue of a subject is believed to be of therapeutic benefit. Examples of such diseases include hyperproliferative diseases and quiescent malignant diseases. In particular embodiments, the disease is a hyperproliferative disease, such as cancer of solid tissues or blood cells. Quiescent malignant diseases that can be treated by MTM or MTM analogue nanoparticles include, for example, chronic lymphocytic leukemia.

For example, MTM an MTM analogue formulated in a pharmaceutically acceptable nanoparticulate formulation can be administered to treat a hyperproliferative disease. The hyperproliferative disease may be cancer, leiomyomas, adenomas, lipomas, hemangiomas, fibromas, pre-neoplastic lesions (such as adenomatous hyperplasia and prostatic intraepithelial neoplasia), carcinoma in situ, oral hairy leukoplakia, or psoriasis.

The cancer may be a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus. In certain embodiments, the cancer is ovarian cancer. In particular aspects, the cancer may be a chemo-resistant cancer, i.e., refractive forms of cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, the present disclosure concerns a method of treating a hyperproliferative disease or a quiescent malignant disease including administering to a subject in need thereof a therapeutically effective amount of the composition including MTM or an MTM SK, SDK or SA analogue or any combination of said molecules formulated in a nanoparticulate formulation comprising self-assembled or cross-linked poly(ethylene glycol)-poly(aspartate hydrazide)-based nanoparticles.

The hyperproliferative disease that can be treated by the method of the present disclosure may be pre-cancer or cancer, such as melanoma, leukemia, ovarian cancer, colon cancer, prostate cancer, lung cancer, liver cancer, pancreatic cancer, bladder cancer, breast cancer, gastric cancer, colon cancer, head and neck cancer, esophagus cancer, synovium cancer, brain cancer, or bronchus cancer, in particular, chronic myelogenous leukemia (CML) or chronic lymphocytic leukemia (CLL). In particular, the disease may be chemo-resistant. The subject to be treated in the present disclosure may be a mammal, such as a human.

### Combination Therapies

In certain embodiments, the compositions and methods of the present disclosure involve MTM analogue nanoparticulate formulation-based composition as set forth herein with a second or additional therapy. Such therapy can be applied in the treatment of any disease for which treatment with the MTM analogue nanoparticulate formulation is contemplated. For example, the disease may be a hyperproliferative disease, such as cancer.

The methods and compositions including combination therapies enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with a therapeutic nucleic acid, such as an inhibitor of gene expression, and a second therapy. A tissue, tumor, or cell can be contacted with one or more compositions or pharmacological formulation(s) including one or more of the agents (i.e., inhibitor of gene expression or an anti-cancer agent), or by contacting the tissue, tumor, and/or cell with two or more distinct compositions or formulations, wherein one composition provides 1) an inhibitor of gene expression; 2) an anti-cancer agent, or 3) both an inhibitor of gene expression and an anti-cancer agent. Also, it is contemplated that such a combination therapy can be used in conjunction with a chemotherapy, radiotherapy, surgical therapy, or immunotherapy.

A therapeutic MTM or MTM analogue nanoparticulate formulation-containing composition set forth herein may be administered before, during, after or in various combinations relative to an anti-cancer treatment. The administrations may be in intervals ranging from concurrently to minutes to days to weeks. In embodiments where MTM or the MTM analogue nanoparticulate formulation-containing composition is provided to a patient separately from an additional anti-cancer agent, one would generally ensure that a significant period of time did not expire between the time of each delivery, such that the two agents would still be able to exert an advantageously combined effect on the patient. In such instances, it is contemplated that one may provide a patient with the inhibitor of gene expression therapy and the anti-cancer therapy within about 12 to 24 or 72 h of each other and, more preferably, within about 6-12 h of each other. In some situations it may be desirable to extend the time period for treatment significantly where several days (2, 3, 4, 5, 6 or 7) to several weeks (1, 2, 3, 4, 5, 6, 7 or 8) lapse between respective administrations.

Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed. For example, a combination therapy in the following pattern or manner may be applied (where a therapeutic MTM or MTM analogue nanoparticulate formulation-containing composition is “A” and an anti-cancer therapy is “B”): A/B/A, B/A/B, B/B/A, A/A/B, A/B/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A or A/A/B/A.

Administration of any compound or therapy of the present disclosure to a patient will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies, as well as radiation and surgical intervention, may be applied in combination with the described therapy.

In specific aspects, it is contemplated that a standard therapy will include chemotherapy, radiotherapy, immunotherapy, surgical therapy or gene therapy and may be employed in combination with the inhibitor of gene expression therapy, anticancer therapy, or both the therapeutic nucleic acid and the anti-cancer therapy, as described herein.

Thus, in certain aspects, to have a better therapeutic benefit, the composition of the present disclosure may be administered in combination with at least an additional agent selected from the group consisting of a radiotherapeutic agent, a hormonal therapy agent, an immunotherapeutic agent, a chemotherapeutic agent, a cryotherapeutic agent and a gene therapy agent.

In particular aspects, the additional agent may be a chemotherapeutic agent. Examples of chemotherapeutic agents include, but are not limited to, cetuximab (erbitux), herceptin (trastuzumab), fludarabine, cyclophosphamide, rituximab, imatinib, Dasatinib (BMS0354825), cisplatin (CDDP), carboplatin, procarbazine, mechlorethamine, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, gemcitabien, navelbine, farnesyl-protein tansferase inhibitors, transplatinum, 5-fluorouracil, vincristin, vinblastin, methotrexate, an analogue or derivative thereof. Particularly, the chemotherapeutic agent may be fludarabine, cyclophosphamide, rituximab, imatinib or Dasatinib. In a certain aspect, the cancer may be resistant to chemotherapy, such as fludarabine, cyclophosphamide, rituximab, imatinib or Dasatinib.

The nanoparticulate formulations of the present disclosure may optionally include one or more additional therapeutic agents. For example, the therapeutic agent may be a chemotherapeutic agent.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBl-TMl); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-II); topoisomerase inhibitor RFS 2000; difluoromethylomithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristine, vinblastine and methotrexate and pharmaceutically acceptable salts, acids or derivatives of any of the above.

Also included in the formulations may be anti-hormonal agents that act to regulate or inhibit hormone action on tumors such as anti-estrogens and selective estrogen receptor modulators (SERMs), including, for example, tamoxifen, raloxifene, droloxifene, A-hydroxytamoxifen, trioxifene, keoxifene, LY1 17018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analogue); antisense oligonucleotides, particularly those which inhibit expression of genes in signaling pathways implicated in abherant cell proliferation, such as, for example, PKC-alpha, RaIf and H-Ras; ribozymes such as a VEGF expression inhibitor and a HER2 expression inhibitor; vaccines such as gene therapy vaccines and pharmaceutically acceptable salts, acids or derivatives of any of the above.

### EXAMPLES

The following examples are included to demonstrate exemplary embodiments of the disclosure. Those of skill in the art should, in light of the present disclosure, appreciate that many changes can be made in the specific embodiments which are disclosed and still obtain a like or similar result without departing from the spirit and scope of the disclosure.

Materials:

L-aspartic acid β-benzyl ester (BLA), triphosgene, anhydrous hydrazine, N,N′-diisopropylcarbodiimide (DIC), N-hydroxysuccinimide (NHS), anhydrous tetrahydrofuran (THF), anhydrous hexane, anhydrous ethyl ether, anhydrous dimethylsulfoxide (DMSO), DMSO (molecular biology grade, ≧99.9%), dimethylsulfoxide-d6 (DMSO-d6), resazurin sodium salt, acetate buffer solution, phosphate buffer solution, adipic acid, sodium hydroxide (NaOH) were purchased from Sigma-Aldrich (USA). α-Methoxy-ω-amino poly(ethylene glycol) (PEG, 5 kDa) was purchased from NOF Corporation (Japan). Regenerated cellulose dialysis bags with molecular weight cut off (MWCO) 6-8 kDa and Slide-A-Lyzer G2 dialysis cassettes with MWCO 10 kDa, methanol (MeOH), acetonitrile (ACN), celite, C18 RP silica gel, tryptic soy broth (TSB), LB broth, Difco agar, sucrose, potassium sulfate, magnesium chloride, glucose, casamino acids, yeast extract, MOPS, and trace elements were purchased from Fisher Scientific (USA). Steptomyces argillaceus ATCC 12956 was purchased from ATCC (USA). SpectraPor 6 dialysis tubing with MWCO 50 kDa was purchased from Spectrum Labs (USA).

### Example 1

### Biosynthesis of MTM Analogues

SK and SDK were produced by an adapted procedure reported previously. Albertini et al., Nucleic Acids Res. 2006; 34(6):1721-1734. Cells of S. argillaceus M7W1 were plated on R5A agar and grown for four days. After four days the spores used to inoculate 100 mL of tryptic soy broth (TSB) for 24 hrs in an orbital shaker at 28° C., 250 rpm. After 24 hrs 4 mL of the TSB culture was used to inoculate 40, 100 mL flasks of R5A media. The cultures were grown for 3 days at 28° C., 250 rpm. After 3 days the cells were harvested and 50 g/L celite was added to the culture. The cells were removed by filtration, re-dissolved in MeOH and sonicated for 1 hr to lyse the cells. After sonication the cells were filtered again and the MeOH was removed from the filtrate, reconstituted in water and loaded onto a 5×12 cm C18 RP column equilibrated with 10 column volumes of water. The column was washed with 10% ACN in water, followed by 20% ACN in water and subsequently eluted with 100% ACN. The filtrate from the initial removal of the cells was also loaded onto the C18 column, followed by the same fractionation procedure. The samples were dried and re-dissolved in 80% MeOH and water. The compounds were further isolated by semi-preparative HPLC.

RESULTS: SK and SDK were successfully produced by the S. argillaceus M7W1, and isolated through an adapted procedure developed previously. S. argillaceus cells of the M7W1 mutant strain were plated on R5A agar and allowed to grow until spores were formed. After two days colonies began to appear on the plates, after three days spores began to form and after four days the majority of the cells were present as spores. Spores from the plate were used to inoculate an initial culture of TSB media. The cells grew quickly in TSB media and were ready to transfer to R5A media after 24 hrs. Once transferred to the R5A growth media the cells were able to produce the drug analogues. Initially the media was adjusted to pH 6.85, however after the observation of the production the pH was adjusted to 7.0. The culture was monitored for the emergence of the MTM analogues using HPLC. After 24 hrs the presence of MTM analogues or precursors were not readily detected. After 48 hrs the MTM analogues began to appear, however a large population of pre-MTM compounds was still observed. After 72 hrs the MTM compounds were the dominant compounds in the culture so the production was stopped. Much of the population of the MTM analogues was excreted in the culture media thus the initial step of the isolation was to separate out the cells from the media by the addition of a celite cell binding resin and subsequent filtration. Once the cells had been removed the filtrate was passed over a C18 RP silica gel column to collect the compounds of interest. The initial washes of 10% and 20% ACN allowed many of the byproducts to be removed from the sample. Further fractionation of the culture on the C18 RP column did not aid in the purification of the individual MTM analogues so it was decided to elute all the MTM compounds together to minimize sample loss and excessive dilution. The cells removed in the initial filtration step did still contain a substantial amount of SK and SDK, thus to take full advantage of the production of the analogues the cultured cells were covered in MeOH and sonicated to lyse the cells and release the drugs. Cellular debris was removed by again filtering the solution which was then able to then be dried, reconstituted in water and loaded on the C18 RP column. After the same wash and elution steps were performed a similar solution to the initial cell filtrate was obtained, however in a smaller quantity. The samples could then be loaded and the individual MTM analogues separated by using semi-preparative HPLC. The individual peaks were collected and analyzed for purity using HPLC-MS. Both the SK and SDK products were collected at >95% purity through this procedure and were stored at −20° C. for future use.

### Example 2

### Block Copolymer Synthesis

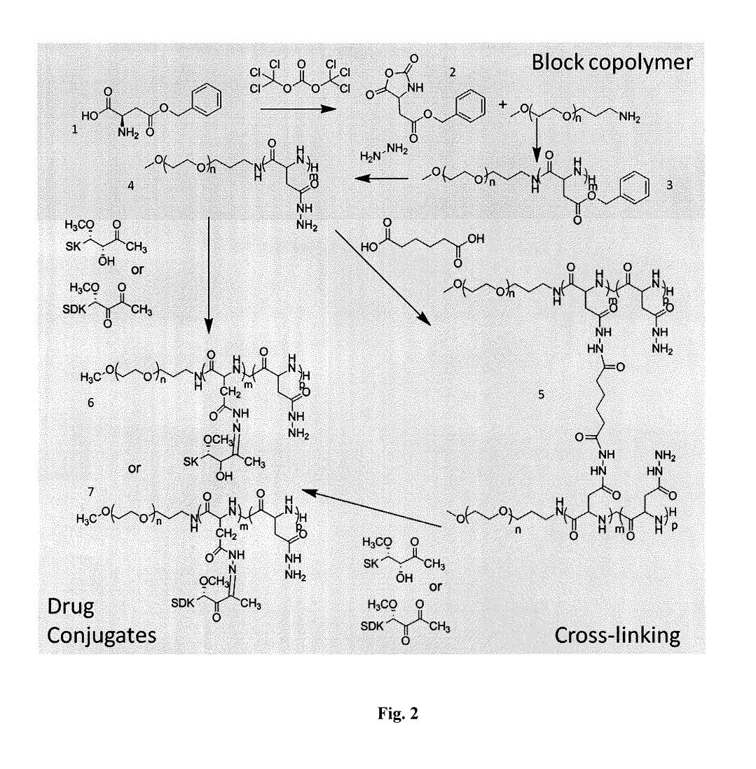

Block copolymers were synthesized as reported previously (FIG. 2). Lee H J et al., Biomacromolecules. Jul. 11, 2011; 12(7):2686-2696, which is hereby incorporated herein by reference. Briefly, β-Benzyl-L-aspartate N-carboxy anhydride (BLA-NCA) was prepared as a monomer by adding 2.88 g of triphosgene to 5.0 g β-benzyl-L-aspartate in 100 mL of dry THF at 45° C. until the solution became clear. Anhydrous hexane was added to the solution slowly to form BLA-NCA crystal, which was recrystallized at −20° C. PEG was used as a macroinitiator to conduct the ring opening polymerization of BLA-NCA, which produced PEG-(β-benzyl L-aspartate) [PEG-PBLA]. BLA-NCA and PEG were reacted in anhydrous DMSO at 50 mg/mL under N2 for 2 days at 45° C. The PEG-PBLA block copolymers were isolated from the reaction mixture by precipitation with anhydrous ethyl ether and subsequently freeze drying. PEG-PBLA (100 mg/mL) was reacted with 20-fold equivalent hydrazine in DMSO (40° C.) for 1 hr to prepare PEG-poly(aspartate hydrazide) block copolymers [PEG-p(Asp-Hyd)]. The materials were collected by ether precipitation and freeze drying. The block copolymer compositions were determined by ¹H-NMR, which is described as X-Y denoting the molecular weight of PEG×10⁻³ (X) and the number of aspartate repeating units (Y), respectively.

RESULTS: ¹H-NMR showed that PEG-p(Asp-Hyd) was successfully synthesized as reported previously, [Lee H J et al., Biomacromolecules. Jul. 11, 2011; 12(7):2686-2696] and used to prepare self-assembled micelles. For cross-linked micelles, the cross-linking of PEG-p(Asp-Hyd) was confirmed by GPC. The cross-linked micelles had an average molecular weight of ˜147 kDa, corresponding to an average of roughly 14 block copolymers per cross-linked micelle. NMR was also used to determine the cross-linking percentage. The carboxyl peak of adipic acid at around 12 ppm disappeared, indicating that no unreacted or partially reacted adipic acid was present in the sample. The cross-linking percentage was 12.5%, which was determined by integrating the newly appeared peaks at 2.1 ppm and 1.5 ppm (α and β CH2 groups of adipic acid) with respect to the PEG peak at 3.5 ppm.

### Example 3

### Synthesis of Self-Assembled Polymer Micelles

MTM analogues were conjugated to the PEG-p(Asp-Hyd) block copolymers by adding both SK and SDK to the block copolymer at 50 weight % in DMSO. The reaction was allowed to proceed at room temperature for 72 hr, shaking at 500 rpm. The materials were collected and purified by repeating ether precipitation. The drug-polymer conjugates were then re-dissolved in deionized water to form self-assembled micelles. The drug-loaded micelles were collected by freeze drying.

### Example 4

### Synthesis of Cross-Linked Polymer Micelles

Cross-linked polymer micelles were created by cross-linking PEG-p(Asp-Hyd) 5-40 with adipic acid. PEG-p(Asp-Hyd) 5-40 was dissolved in DMSO to a final concentration of 100 mg/mL. Adipic acid, NHS, and DIC were combined in DMSO to a mole ratio of 0.5:4:4 times the hydrazide groups of the block copolymer, respectively, and the reaction mixture was added the block copolymer. The cross-linking reaction was conducted for 72 hrs, stirring at room temperature. The reaction solution was then dialyzed against DMSO, followed by 50% DMSO, and finally DI water. The solution was freeze dried to collect the purified materials. The cross-linked micelles were characterized by gel permeation chromatography (GPC) and ¹H-NMR. MTM analogues were conjugated to cross-linked micelles at a 50 weight % in DMSO, followed by ether precipitation and freeze drying.

### Example 5

### Micelle Characterization

Drug loading efficiency in polymer micelles was determined by quantifying the absorbance of MTM analogues at 410 nm. Particle size of the micelles was determined by dynamic light scattering (DLS) on a Zetasizer Nano-ZS (Malvern, UK) equipped with a He—Ne laser light source and 173° angle scattered light collection configuration.

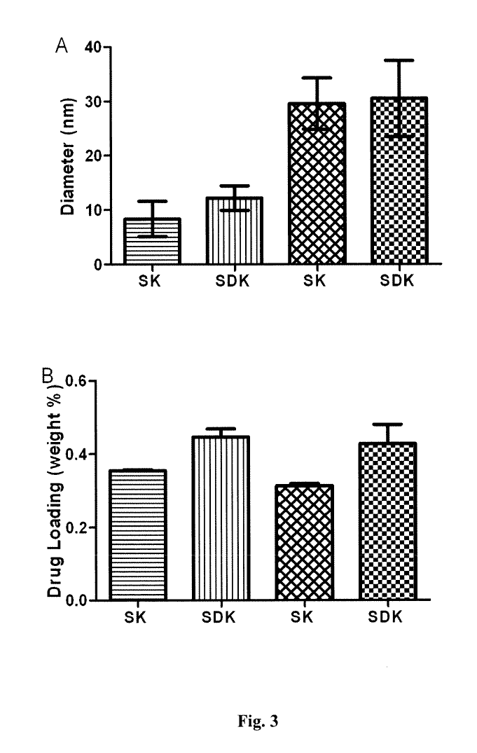

RESULTS: Following the drug conjugation reaction, ether precipitation was used to collect the polymers. Initial precipitations possessed a yellow supernatant corresponding to free drug that was not entrapped in the micelle structures. Ether precipitations were continued until the supernatant remained clear for several precipitations. The polymers maintained a yellowish color, after free drugs were removed, indicating the presence of the MTM analogues. UV-VIS absorption spectrometry at 410 nm confirmed that both SK and SDK were successfully entrapped in both the self-assembled micelles and cross-linked micelles. Drug loading in the self-assembled micelles was 35.4±0.3 and 44.6±2.2 weight % (wt %) for SK and SDK, respectively. Cross-linked micelles had an average drug loading of 31.3±0.3 and 42.7±5.3 wt % for SK and SDK, respectively. DLS showed that the particle size of self-assembled micelles entrapping SK and SDK was 8.36±3.21 and 12.19±2.77 nm. The particle size of cross-linked micelles was 29.56±4.67 nm for SK and 30.48±7.00 nm for SDK (FIG. 3).

### Example 6

### Drug Release Experiments

Drug release patterns of the self-assembled or cross-linked polymer micelles were evaluated by using dialysis cassettes (Slide-A-Lyzer G2, MWCO 10 kDa, Thermo Scientific, USA). Dialysis cassettes containing 2 mL samples were placed in 4 L of phosphate buffer solution (10 mM, pH 7.4 or 5.0). The temperature was maintained at 37° C. An aliquot of 150 μL was taken out of each dialysis cassette at the time intervals of 0, 0.5, 1, 3, 6, 24, 48, and 72 hrs. The aliquots taken at each time point were analyzed by UV/Vis absorbance at 410 nm. All experiments were performed in triplicate.

RESULTS: A pH sensitive hydrazone linkage was used to conjugate MTM analogues to the block copolymers to take advantage of the acidic tumor environment. To test pH-responsive drug release patterns, MTM nanoparticulate formulations were incubated at pH 7.4 and 5.0. FIG. 4 shows that all MTM analogue nanoparticulate formulations released drugs effectively. At pH 7.4, self-assembled micelles entrapped more than 50% of MTM analogues for 24 hrs, which will be stable enough to deliver drugs to tumor tissues following intravenous injections. Drug release from the self-assembled micelles was accelerated at pH 5.0, suggesting that SK and SDK will be released effectively in acidic tumor tissues. Cross-linked micelles appeared to release drugs more quickly at pH 7.4 than self-assembled micelles, yet retained 40% of drugs for 24 hrs. Both SK and SDK were released more rapidly from cross-linked micelles at pH 5.0.

### Example 7

### Cell Toxicity Assays in A549 Cells

The cytotoxic effects of four MTM analogue nanoparticulate formulations were investigated in comparison to free drugs. A549 cells were cultured as specified from ATCC at 37° C., 5% CO2. The cells were seeded on a 96 well plate (5,000 cells/well) and allowed to adhere for 24 hrs. After 24 hrs growth media were replaced with fresh media containing samples at differing concentrations. 1% DMSO (biotechnological grade) was used as a control vehicle to prepare free drug formulations for SK and SDK. Self-assembled and cross-linked micelles were used as nanoparticulate formulations for SK and SDK. The cells were exposed to samples for 72 hrs total (n=8). Cell viability was assessed using a resazurin assay that indicates mitochondrial metabolic activity in live cells. 10 μL of a 1 mM resazurin solution in PBS was added to the vehicle- and drug-treated cells at the end of the treatment period. Cell viability was determined three hours later by reading the fluorescence at 560 nm (Ex)/590 nm (Em). The fluorescence signals were quantified using a Spectramax M5 plate reader (Molecular Devices) equipped with a SoftMaxPro software. Cytotoxicity was determined by calculating the half maximal inhibitory concentration (IC50) of each sample.

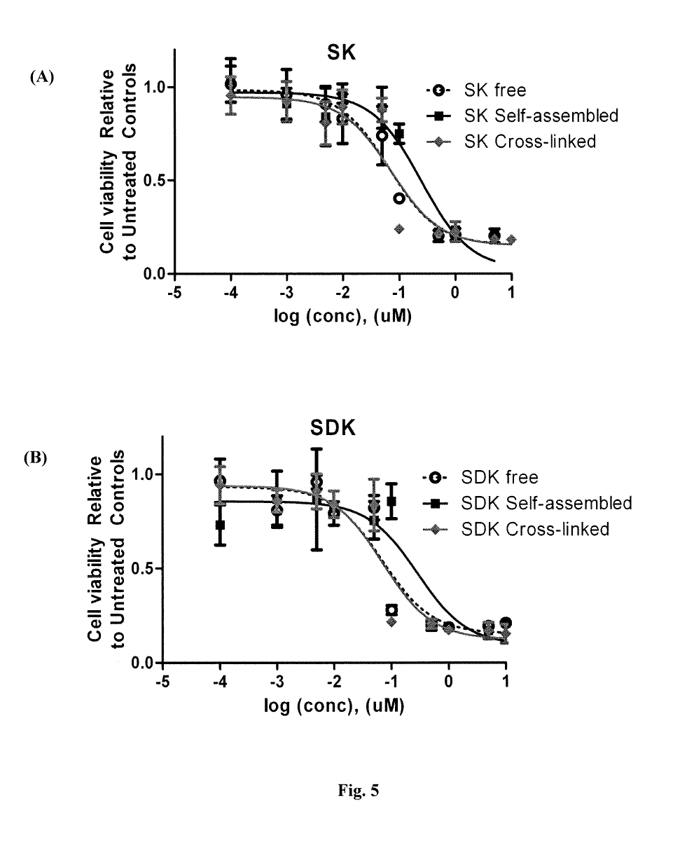

RESULTS: Cytotoxicity of MTM analogue nanoparticulate formulations was tested with A549 cells (FIG. 5). Free SK and SDK showed IC50 values of 0.163±0.092 μM and 0.093±0.035 μM, respectively. IC50 values of self-assembled micelles entrapping MTM analogues were 0.262±0.087 μM and 0.282±0.131 μM for SK and SDK, which were 1.6 and 3 fold higher than those of free drugs. Interestingly, cross-linked micelles showed IC50 values of 0.073±0.015 μM and 0.075±0.009 μM for SK and SDK, which were 2.2 and 1.2 fold lower than free drugs (Table 1). No toxicity was observed with block copolymers, self-assembled micelles and cross-linked micelles alone up to 100 μM (data not shown).

TABLE 1
A summary of the characteristics and activity of the drug loaded polymer nano-particles
and that of the free drugs for comparison.

| | SK | | | SDK | | |
|---|---|---|---|---|---|---|
| | | Self- | Cross- | | Self- | Cross- |
| Drug | | assembled | linked | | assembled | linked |
| Formulation | Free | Micelles | Micelles | Free | Micelles | Micelles |
| IC50 (nM) | 163 ± 92 | 262 ± 87 | 73 ± 15 | 93 ± 36 | 282 ± 131 | 75 ± 9 |
| Relative | 1.00 | 0.62 | 2.23 | 1.00 | 0.33 | 1.24 |
| efficacy to free | | | | | | |
| drug | | | | | | |

### Example 8

### Cell Toxicity Assays in HT29 Cells

The in vitro cytotoxic effects of the MTM loaded CNAs (Crosslinked Nano Assemblies) were investigated in comparison to free drugs. HT29 cells were cultured as specified from ATCC at 37° C., 5% CO₂. The cells were seeded on a 96 well plate (5,000 cells/well) and allowed to adhere for 24 hrs. After 24 hrs growth media were replaced with fresh media containing drug and CNA samples at differing concentrations. Free drug formulations for SK and SDK and CNA formulations were prepared in filtered DI water. The cells were exposed to samples for 72 hrs total (n=4). Cell viability was assessed using a resazurin assay that indicates mitochondrial metabolic activity in live cells. 10 μL of a 1 mM resazurin (7-Hydroxy-3H-phenoxazin-3-one 10-oxide) solution in PBS was added to the vehicle- and drug-treated cells at the end of the treatment period. Cell viability was determined three hours later by reading the fluorescence at 560 nm (Ex)/590 nm (Em). The fluorescence signals were quantified using a Spectramax® M5 plate reader (Molecular Devices) equipped with a SoftMaxPro® software. Cytotoxicity was determined by calculating the half maximal inhibitory concentration (IC50) of each sample.

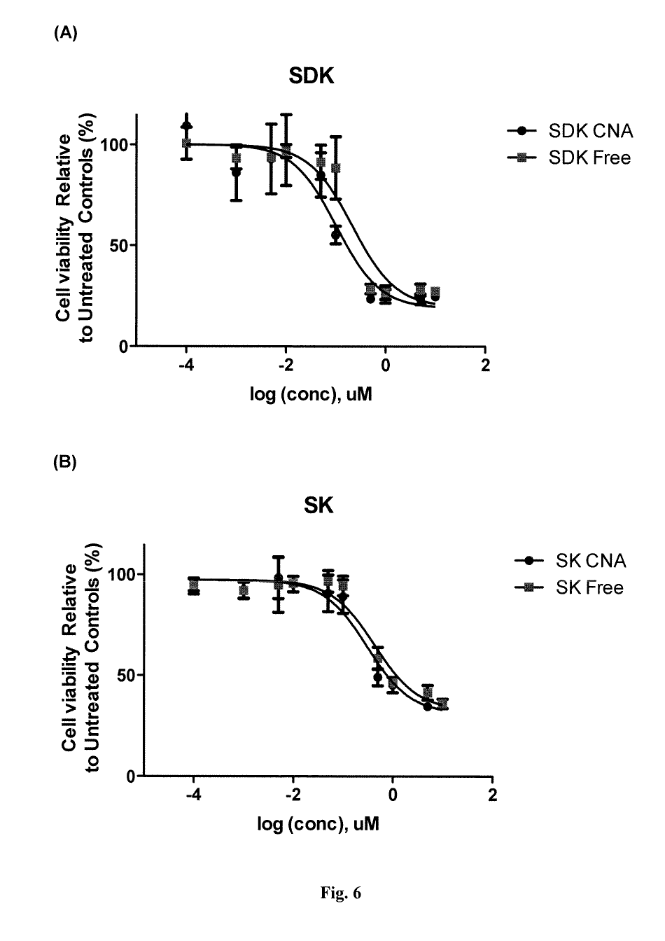

RESULTS: Cytotoxicity of MTM loaded CNAs and MTM free drugs were tested with HT-29 cells (FIG. 6). Free SK and SDK showed IC50 values of 0.429±0.003 μM and 0.216±0.004 μM, respectively. IC50 values of CNAs entrapping MTM analogues were 0.305±0.003 μM and 0.103±0.005 μM for SK and SDK, respectively. The IC50 values of the CNA formulations showed the CNAs to be 29% more effective in the case of the SK CNA and 52% more effective in the case of the SDK CNA. No toxicity was observed with CNAs alone up to 100 μM (data not shown).

### Example 9

### In Vivo Tumor Model

Subcutaneous tumor models were established with HT29 colon cancer cells. Nude/nude mice at 8 weeks old were injected with 3×106 HT29 cells subcutaneously in the right flank. The tumors were then allowed to grow to a volume of ˜130 mm³. Once the tumors reached a volume of 130 mm³ the mice were injected with 100 μL of the desired treatment. For the control group the mice were injected with PBS. For the SK free drug and SK CNA groups the final treatment dose was 7 mg/kg and an injection volume of 100 μL. For SDK free drug and SDK CNA the final treatment dose was 2 mg/kg with an injection volume of 100 μL. Once the mice were treated the tumor volume and body mass were measured for 18 days. The groups were formed with an N=6 mice. The tumor volume was calculated by the formula 0.5(l)×w2.

RESULTS: The treatment with free SK resulted in only a 3.5% regression in tumor volume as compared to the control while the treatment with the SK CNA demonstrated a 36% regression in tumor volume as compared to the control after 18 days. For the SDK free drug and CNA formulations demonstrated a relative tumor regression of 48% and 55% as compared to the control, respectively. See FIG. 7.

The above description is provided to enable any person skilled in the art to practice the various aspects described herein. The above description provides various examples of the subject technology, and the subject technology is not limited to these examples. Various modifications to these aspects will be readily apparent to those skilled in the art, and the generic principles defined herein may be applied to other aspects.

All patents and/or publications including journal articles cited in this disclosure are expressly incorporated herein by reference. The disclosure has been described with reference to various specific and preferred embodiments and techniques. However, it should be understood that many variations and modifications may be made while remaining within the spirit and scope of the disclosure.

What is claimed is:

1. A nanoparticulate formulation comprising:
(i) mithramycin (MTM) or one or more MTM analogues selected from the group consisting of SK (MTM with short side-chain and a ketone), SDK (MTM with short side-chain and diketone), SA (MTM with short side-chain and carboxylic acid) and a derivative thereof, as load; and
(ii) block copolymer units of a polyamide and a biocompatible polymer as carrier.

2. The formulation of claim 1, wherein the polyamide comprises amino acid units derived from amino acids with side chains that can have positive, neutral, or negative charges.

3. The formulation of claim 1, wherein the biocompatible polymer is selected from the group consisting of poly(ethylene glycol) (PEG), chitosan (CS), polyethylenimine (PEI), poly(L-lactic acid) (PLLA), poly(D-lactic acid) (PDLA), poly(2-hydroxy ethyl methacrylate) (PHEMA), poly(ε-caprolactone) (PCL), poly(vinyl acetate) (PVAc), poly(ethylene oxide) (PEO), poly[(R)-3-hydroxybutyric acid)] (PHB), cellulose acetate (CA), poly(lactide-co-glycolide) (PLGA), poly(N-isopropylacrylamide) (PNIPAM), polyalkyleneglycol, polyalkyleneoxide, polyvinylpyrrolidone, polysaccharide, polyacrylamide, polymethacrylamide, polyvinylalcohol and derivatives thereof.

4. The formulation of claim 1, wherein the copolymer units form nanoparticles in form of supermolecular structures selected from the group consisting of micelles, vesicles, layers, membrane, sphere, aggregate, tube, fiber, ribbon and sheet.

5. The formulation of claim 2, wherein one or more of the amino acid units comprises a pH-sensitive linkage in its side chain, wherein said pH-sensitive linkage is selected from the group consisting of acetal, orthoester, cis-aconityl group, hydrazone, imine, ester, Schiff base, dithioacetal, tert butyl ester, carbamate, thioester, or phosphoramidate.

6. The formulation of claim 5, wherein the amino acid units comprises aspartic acid or glutamic acid or both.

7. The formulation of claim 5, wherein the MTM or one or more of the MTM analogues is conjugated to one or more of the amino acid units via the pH-sensitive linkage.

8. The formulation of claim 7, wherein the pH-sensitive linkage is stable at a pH between about 7 and 8 and is hydrolyzed at a pH less than about 7 to release the MTM or one or more of the MTM analogues.

9. The formulation of claim 1, wherein the block copolymer is poly(ethylene glycol)-poly(aspartate hydrazide) copolymer.

10. The formulation of claim 1, wherein the block copolymer units self-assemble or crosslink to form a micelle with the polyamide forming the core and the biocompatible polymer forming the corona of the micelle.

11. The formulation of claim 1, wherein the formulation is freeze dried to form a freez-dried formulation.

12. A method of treating a hyperproliferative disease or a quiescent malignant disease comprising administering to a subject in need thereof a therapeutically effective amount of a nanoparticulate formulation comprising:
(i) mithramycin (MTM) or one or more MTM analogues selected from the group consisting of SK, SDK, SA and a derivative thereof, as load; and
(ii) block copolymer units of a polyamide and a biocompatible polymer as carrier.

13. The method of claim 12, wherein the polyamide comprises amino acid units derived from amino acids with side chains that can have positive, neutral, or negative charges.

14. The method of claim 12, wherein the biocompatible polymer is selected from the group consisting of poly(ethylene glycol) (PEG), chitosan (CS), polyethylenimine (PEI), poly(L-lactic acid) (PLLA), poly(D-lactic acid) (PDLA), poly(2-hydroxy ethyl methacrylate) (PHEMA), poly(ε-caprolactone) (PCL), poly(vinyl acetate) (PVAc), poly(ethylene oxide) (PEO), poly[(R)-3-hydroxybutyric acid)] (PHB), cellulose acetate (CA), poly(lactide-co-glycolide) (PLGA), poly(N-isopropylacrylamide) (PNIPAM), polyalkyleneglycol, polyalkyleneoxide, polyvinylpyrrolidone, polysaccharide, polyacrylamide, polymethacrylamide, polyvinylalcohol and derivatives thereof.

15. The method of claim 12, wherein the copolymer units form nanoparticles in form of supermolecular structures selected from the group consisting of micelles, vesicles, layers, membrane, sphere, tube, fiber, ribbon and sheet.

16. The method of claim 15, wherein one or more of the amino acid units comprises a pH-sensitive linkage in its side chain, wherein said pH-sensitive linkage is selected from the group consisting of acetal, orthoester, cis-aconityl group, hydrazone, imine, ester, Schiff base, dithioacetal, tert butyl ester, carbamate, thioester, or phosphoramidate.

17. The method of claim 16, wherein the amino acid units comprises aspartic acid or glutamic acid or both.

18. The method of claim 16, wherein the MTM or one or more of the MTM analogues is conjugated to one or more of the amino acid units via the pH-sensitive linkage.

19. The method of claim 18, wherein the pH-sensitive linkage is stable at a pH between about 7 and 8 and is hydrolyzed at a pH less than about 7 to release the MTM or one or more of the MTM analogues.

20. The method of claim 12, wherein the block copolymer is poly(ethylene glycol)-poly(aspartate hydrazide) copolymer.
